(19) United States
(12) Patent Application Publication
(10) Pub. No.: US 2026/0306248 A1
(43) Pub. Date: Oct. 8, 2026

(54) SYSTEMS FOR AMNIOTIC TISSUE OCULAR SURFACE RESURFACING AND METHODS THEREFOR

(71) Applicant: Restore Vision Partners, LP, Amarillo, TX (US)

(72) Inventor: Blake Smith, Amarillo, TX (US)

(73) Assignee: Restore Vision Partners, LP, Amarillo, TX (US)

(21) Appl. No.: 19/714,362

(22) Filed: Jun. 19, 2026

Related U.S. Application Data
(63) Continuation-in-part of application No. 19/182,905, filed on Apr. 18, 2025.
(60) Provisional application No. 63/738,144, filed on Dec. 23, 2024.

(51) Int. Cl. A61F 2/14; A61L 27/36

(52) U.S. Cl. CPC A61F 2/142; A61F 2/148; A61L 27/3604; A61F 2210/0076; A61F 2220/0008; A61L 2430/16

(57) ABSTRACT

An ocular surface resurfacing system includes an amniotic tissue body that is laid onto a corneal surface of an eye and conforms to and fills one or more surface irregularities of the corneal surface to present a smooth ocular surface, and a securing element that retains the amniotic tissue body against the corneal surface. The amniotic tissue body may comprise a non-handed construction presenting equivalent amniotic tissue to the corneal surface independent of which face contacts the corneal surface, and may be retained by a native tackiness developed by removing moisture from the corneal surface without applying a saline. The corneal surface may be resurfaced without debriding the corneal surface, such that a corneal epithelium is preserved, to optimize the corneal surface for a biometric measurement or a corneal mapping obtained prior to an ocular surgery.

### CROSS-REFERENCE TO RELATED APPLICATIONS

The present application is a Continuation in Part of U.S. Patent Application No. 19/182,905, filed Apr. 18, 2025, entitled “Amniotic Membrane Method of Use,” which claims priority to U.S. Provisional Patent Application No. 63//738,144, filed Dec. 23, 2024, the entireties of which are hereby incorporated by reference for all purposes.

### TECHNICAL FIELD

The present disclosure relates generally to ocular resurfacing, and more particularly to systems and methods for resurfacing a corneal surface to present a smooth ocular surface for ocular surgery using amniotic tissues.

### BACKGROUND

Ocular surgeries, including cataract surgery and laser-refractive procedures, depend upon an accurate measurement of an eye and of a corneal surface of the eye. A biometric measurement, a corneal topography, and/or an intraocular lens calculation obtained prior to an ocular surgery may be derived, at least in part, from an ocular surface presented by the corneal surface. Where the corneal surface presents one or more surface irregularities, an instability, and/or a dryness, the ocular surface presented by the corneal surface may be distorted, and a measurement obtained from the corneal surface may be subject to an error. Such an error may propagate into a selection of an intraocular lens, a planning of a laser ablation, and/or an outcome of an ocular surgery, such that an irregularity of the corneal surface may contribute to a suboptimal visual outcome notwithstanding an otherwise technically successful surgery.

Various approaches have been employed in an effort to address a condition of the corneal surface prior to an ocular surgery. For example, an artificial tear and/or a topical agent may be applied to the corneal surface to address a symptom of a dryness, and, in some approaches, a corneal surface may be prepared by a removal and/or a scraping of a tissue of the corneal surface. Amniotic tissue has been applied to an ocular surface in connection with treating one or more ocular surface conditions. However, an approach directed to a symptom of a condition of the corneal surface may not restore the corneal surface to a smooth ocular surface suitable for an accurate measurement, and an approach involving a removal and/or a scraping of a tissue of the corneal surface may disturb the corneal surface.

Accordingly, current solutions are not robust enough to restore a corneal surface to a smooth ocular surface from which an accurate measurement may be obtained prior to an ocular surgery, especially while preserving a tissue of the corneal surface, or to implement the solution in a manner suitable for a clinical setting. The present disclosure addresses these challenges, as well as one or more additional considerations, as described herein.

### BRIEF SUMMARY

The present disclosure achieves technical advantages as systems and methods for an ocular surface resurfacing system and techniques for resurfacing a corneal surface to present a smooth ocular surface for ocular surgery. In particular embodiments, an ocular surface resurfacing system may be configured with functionality to conform amniotic tissue to a corneal surface so as to fill surface irregularities of the corneal surface, to retain the amniotic tissue against the corneal surface, and/or to deliver regenerative and/or anti-inflammatory factors to the corneal surface. In embodiments, the ocular surface resurfacing system may be configured to optimize the corneal surface for a biometric measurement and/or a corneal mapping obtained prior to an ocular surgery, and, in embodiments, may be configured to treat dry eye. These functionalities of the ocular surface resurfacing system of embodiments may operate to address the ocular and biometric consequences of an irregular corneal surface as described herein.

In embodiments, the ocular surface resurfacing system may be configured to lay an amniotic tissue body onto the corneal surface such that the amniotic tissue body conforms to and fills one or more surface irregularities of the corneal surface so as to present a smooth ocular surface, in a manner of filling one or more potholes of the corneal surface. By presenting a smooth ocular surface, the ocular surface resurfacing system may be configured to reduce an error that the one or more surface irregularities may otherwise propagate into a corneal topography, a biometric measurement, and/or an intraocular lens calculation obtained from the corneal surface prior to an ocular surgery, and to optimize the corneal surface for a biometric measurement and/or a corneal mapping obtained prior to the ocular surgery. In this manner, the ocular surface resurfacing system of embodiments may be configured to resurface the corneal surface for the object of presenting a smooth ocular surface from which a measurement may be obtained, as distinct from a use of an amniotic tissue as a passive patch over the corneal surface.

In embodiments, the ocular surface resurfacing system may be configured to retain the amniotic tissue body against the corneal surface by way of a native developed-tackiness interface formed by removing moisture from the corneal surface, such that the amniotic tissue body may be retained against the corneal surface by a dry adhesion and without an application of a saline and/or a rehydrating solution to the corneal surface. In embodiments, the amniotic tissue body may comprise a non-handed construction configured to present equivalent amniotic tissue to the corneal surface independent of which face of the amniotic tissue body is placed against the corneal surface, such that the amniotic tissue body may be laid onto the corneal surface without orienting a particular face of the amniotic tissue body toward the corneal surface. In embodiments, the amniotic tissue body may be laid onto the corneal surface without debriding and/or scraping the corneal surface and/or a corneal epithelium thereof, such that the corneal epithelium may be preserved beneath the amniotic tissue body. It is noted that, in embodiments, the ocular surface resurfacing system may be implemented in any of a plurality of exemplary forms and/or embodiments, including a securing element provided as a bandage contact lens, an adhesive interface, the native developed-tackiness interface, and/or a therapeutic-eluting lens, an amniotic tissue body provided in a cryopreserved, dehydrated, and/or hydrated form and/or as an orientation-independent construction, and/or one or more alternative modes of delivery of an amniotic tissue to the corneal surface, as described in greater detail herein.

It is an object of the disclosure to provide a method of resurfacing a corneal surface to present a smooth ocular surface for ocular surgery. It is a further object of the disclosure to provide a system for resurfacing a corneal surface to present a smooth ocular surface for ocular surgery. These and other objects are provided by the present disclosure, including at least the following embodiments.

In one particular embodiment, a method of resurfacing a corneal surface to present a smooth ocular surface for ocular surgery is provided. The method includes developing a native tackiness of the corneal surface by removing moisture from the corneal surface, without applying a saline or a rehydrating solution to the corneal surface, such that a corneal epithelium of the corneal surface develops a tacky consistency and laying an amniotic tissue body onto the corneal surface such that the amniotic tissue body conforms to and fills one or more surface irregularities of the corneal surface so as to present a smooth ocular surface. In embodiments, the amniotic tissue body comprises a non-handed construction that presents equivalent amniotic tissue to the corneal surface independent of which face of the amniotic tissue body is placed against the corneal surface, and the amniotic tissue body is laid onto the corneal surface without orienting a stromal side of the amniotic tissue body toward the corneal surface and without debriding or scraping the corneal surface or the corneal epithelium, such that the corneal epithelium is preserved beneath the amniotic tissue body. The method also includes retaining the amniotic tissue body against the corneal surface, at least in part by the tacky consistency of the corneal epithelium or by a securing element placed over the amniotic tissue body, allowing the amniotic tissue body to dwell upon the corneal surface over a dwell period during which a regenerative or anti-inflammatory factor of the amniotic tissue body is delivered to the corneal surface, and obtaining a biometric measurement or a corneal mapping from the smooth ocular surface presented by the amniotic tissue body for the ocular surgery.

In another embodiment, a system for resurfacing a corneal surface to present a smooth ocular surface for ocular surgery is provided. The system comprises an amniotic tissue body configured to be laid onto a corneal surface of an eye and to conform to and fill one or more surface irregularities of the corneal surface so as to present a smooth ocular surface, the amniotic tissue body comprising a non-handed construction configured to present equivalent amniotic tissue to the corneal surface independent of which face of the amniotic tissue body is placed against the corneal surface, and a securing element configured to retain the amniotic tissue body against the corneal surface. In embodiments, the ocular surface resurfacing system is configured to resurface the corneal surface without debriding or scraping the corneal surface or a corneal epithelium thereof, such that the corneal epithelium is preserved beneath the amniotic tissue body.

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims, if any. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings, in which:

FIG. 1 shows an exemplary ocular surface resurfacing system configured with capabilities and functionality for resurfacing a corneal surface to present a smooth ocular surface in accordance with embodiments of the present disclosure.

FIGS. 2A–2D show cross-sectional views of various exemplary forms of the amniotic tissue body of the ocular surface resurfacing system implemented in accordance with embodiments of the present disclosure.

FIGS. 3A-3E show cross-sectional views, taken at an interface between the amniotic tissue body and the corneal surface, of various examples of the securing element of the ocular surface resurfacing system implemented in accordance with embodiments of the present disclosure.

FIG. 4 shows a cross-sectional view of a therapeutic-eluting lens of the ocular surface resurfacing system implemented in accordance with embodiments of the present disclosure.

FIGS. 5A and 5B show an example of a magnified surface profiles of the corneal surface of the eye before and after resurfacing by the ocular surface resurfacing system in accordance with embodiments of the present disclosure.

FIGS. 6A-6E show the ocular surface resurfacing system at successive stages of operation upon the corneal surface of the eye in accordance with embodiments of the present disclosure.

FIGS. 7A and 7B show the ocular surface resurfacing system during alternative modes of delivery of an amniotic tissue to the corneal surface of the eye in accordance with embodiments of the present disclosure.

FIG. 8 shows a high-level flow diagram of a method of preparing a corneal surface for an ocular surgery in accordance with embodiments of the present disclosure.

FIG. 9 shows a high-level flow diagram of a diagnosis and evaluation phase for evaluating a condition of the corneal surface in accordance with embodiments of the present disclosure.

FIGS. 10A-10D show high-level flow diagrams of application procedures for applying an amniotic tissue to the corneal surface in accordance with embodiments of the present disclosure.

FIG. 11 shows a high-level flow diagram of a follow-up and evaluation process for evaluating a condition of the corneal surface following application of an amniotic tissue in accordance with embodiments of the present disclosure.

FIG. 12 is a high-level flow diagram of a pre-surgical biometric optimization phase for optimizing the corneal surface for a biometric measurement prior to an ocular surgery in accordance with embodiments of the present disclosure.

FIG. 13 is a high-level flow diagram of a severe-trauma application process for applying an amniotic tissue to a corneal surface having a severe corneal trauma in accordance with embodiments of the present disclosure.

FIG. 14 is a high-level flow diagram of a laser-refractive preoperative preparation process for preparing a corneal surface for a laser-refractive procedure in accordance with embodiments of the present disclosure.

FIG. 15 is a high-level flow diagram of a laser-refractive postoperative corneal healing process for promoting a healing of a corneal surface following a laser-refractive procedure in accordance with embodiments of the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION

The disclosure presented in the following written description and the various features and advantageous details thereof, are explained more fully with reference to the non-limiting examples included in the accompanying drawings and as detailed in the description. Descriptions of well-known components have been omitted to not unnecessarily obscure the principal features described herein. The examples used in the following description are intended to facilitate an understanding of the ways in which the disclosure can be implemented and practiced. A person of ordinary skill in the art would read this disclosure to mean that any suitable combination of the functionality or exemplary embodiments below could be combined to achieve the subject matter claimed. The disclosure includes either a representative number of species falling within the scope of the genus or structural features common to the members of the genus so that one of ordinary skill in the art can recognize the members of the genus. Accordingly, these examples should not be construed as limiting the scope of the claims.

A person of ordinary skill in the art would understand that any system claims presented herein encompass all of the elements and limitations disclosed therein, and as such, require that each system claim be viewed as a whole. Any reasonably foreseeable items functionally related to the claims are also relevant. The Examiner, after having obtained a thorough understanding of the disclosure and claims of the present application has searched the prior art as disclosed in patents and other published documents, i.e., nonpatent literature. Therefore, the issuance of this patent is evidence that: the elements and limitations presented in the claims are enabled by the specification and drawings, the issued claims are directed toward patent-eligible subject matter, and the prior art fails to disclose or teach the claims as a whole, such that the issued claims of this patent are patentable under the applicable laws and rules of this jurisdiction.

Various embodiments of the present disclosure are directed to an ocular surface resurfacing system and to techniques for resurfacing a corneal surface to present a smooth ocular surface for ocular surgery. In embodiments, an ocular surface resurfacing system may be configured with functionality to conform amniotic tissue to a corneal surface so as to fill surface irregularities of the corneal surface, to retain the amniotic tissue against the corneal surface, and/or to deliver regenerative and/or anti-inflammatory factors to the corneal surface. In embodiments, the ocular surface resurfacing system may be configured to optimize the corneal surface for a biometric measurement and/or a corneal mapping obtained prior to an ocular surgery, and, in embodiments, may be configured to treat dry eye. These functionalities of the ocular surface resurfacing system of embodiments may operate to address the ocular and biometric consequences of an irregular corneal surface as described herein.

It is noted that, in embodiments, a smooth ocular surface may include a smooth corneal surface. For example, a smooth corneal surface may include a surface of the cornea (e.g., including the epithelial surface, the superficial stroma, etc.) that excludes areas of dryness over the corneal surface, that excludes undulations or waviness, that excludes defects, that excludes heaps of epithelium, that excludes areas of exposure of the stroma, excludes abrasions, erosions, ulcers and/or elevated scars, and/or that excludes any other irregularities of the corneal surface. The foregoing characteristics of a smooth corneal surface are provided for exemplary purposes, and should not be construed as limiting in any way. Indeed, in some embodiments, a smooth corneal surface may include other characteristics not explicitly described herein and that those of ordinary skill in the art would understand.

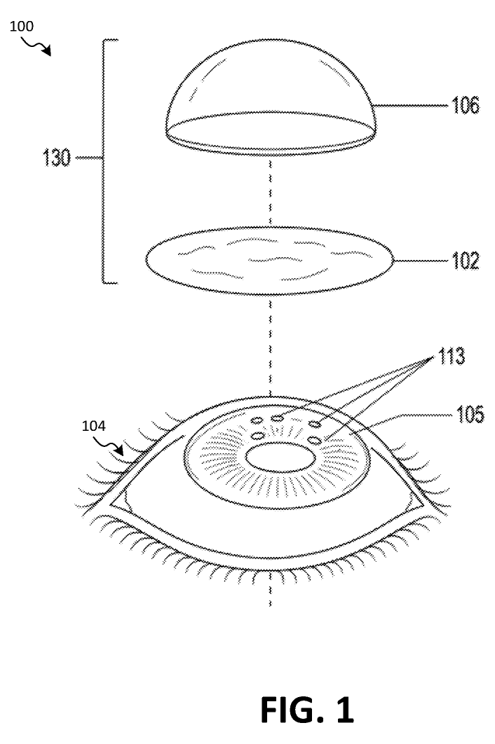

FIG. 1 shows an exemplary ocular surface resurfacing system 100 configured with capabilities and functionality for resurfacing a corneal surface 105 to present a smooth ocular surface in accordance with embodiments of the present disclosure. As shown in FIG. 1, the ocular surface resurfacing system 100 may include an amniotic tissue body 102 and a securing element 130 (shown in an exploded relation to an eye 104 having a cornea and a corneal surface 105). In embodiments, the corneal surface 105 may present one or more surface irregularities 113, and the ocular surface resurfacing system 100 may be configured to be applied to the corneal surface 105 to fill the one or more surface irregularities 113. It is noted that, in embodiments, the eye 104, the corneal surface 105, a corneal epithelium 107, a tear film 109, an eyelid 111, and the one or more surface irregularities 113 described herein with reference to the figures represent an anatomical environment in which the ocular surface resurfacing system 100 operates, and in some embodiments do not form a part of the ocular surface resurfacing system 100.

In embodiments, the ocular surface resurfacing system 100 may be configured to lay the amniotic tissue body 102 onto the corneal surface 105 such that the amniotic tissue body 102 conforms to and fills the one or more surface irregularities 113 of the corneal surface 105 to present a smooth ocular surface, and to retain the amniotic tissue body 102 against the corneal surface 105 by way of the securing element 130. In embodiments, the ocular surface resurfacing system 100 may be configured to deliver regenerative and/or anti-inflammatory factors of the amniotic tissue body 102 to the corneal surface 105 over a dwell period. The ocular surface resurfacing system 100 may be advantageous in that, by filling the one or more surface irregularities 113 to present a smooth ocular surface, the ocular surface resurfacing system 100 may be configured to improve the recovery results following an ocular surgery and/or to reduce the measurement error that the one or more surface irregularities 113 may otherwise introduce into a biometric measurement, a corneal topography, and/or an intraocular lens calculation obtained from the corneal surface 105 prior to an ocular surgery.

In embodiments, the ocular surface resurfacing system 100 may be configured for use across a plurality of clinical contexts. For example, in embodiments, the ocular surface resurfacing system 100 may be configured for use in a cataract-preoperative context, in which the corneal surface 105 is resurfaced to present a smooth ocular surface for a biometric measurement and/or an intraocular lens calculation prior to a cataract surgery and/or to improve the recovery results following the cataract surgery. In another example, the ocular surface resurfacing system 100 may be configured for use in in a laser-refractive context, including a pre-operative sub-context in which the corneal surface 105 is resurfaced to present a smooth ocular surface for a corneal mapping prior to a laser-refractive procedure and/or in a post-operative sub-context in which the amniotic tissue body 102 is applied to the corneal surface 105 to promote healing following a laser-refractive procedure. In still another example, the ocular surface resurfacing system 100 may be configured for use in a standalone dry-eye context, in which the ocular surface resurfacing system 100 is applied to the corneal surface 105 to treat dry eye. It is noted that the foregoing clinical contexts are exemplary and should not be construed as limiting in any way, and the ocular surface resurfacing system 100 may be configured for use in any suitable clinical context in which resurfacing of the corneal surface 105 and/or delivery of regenerative and/or anti-inflammatory factors to the corneal surface 105 may be desirable.

In embodiments, the amniotic tissue body 102 may be configured to operate as the principal operative article of the ocular surface resurfacing system 100, and the securing element 130 (which may be optional in some embodiments) may be configured to retain the amniotic tissue body 102 against the corneal surface 105. As shown in FIG. 1, the securing element 130 may be instantiated as a bandage contact lens 106 configured to be placed over the amniotic tissue body 102 and the corneal surface 105. It is noted that various exemplary forms of the amniotic tissue body 102 are described in greater detail herein, as well as various examples of the securing element 130.

In embodiments, the ocular surface resurfacing system 100 may comprise the amniotic tissue body 102 and the securing element 130, and may further comprise one or more instruments and/or agents configured to cooperate with the amniotic tissue body 102 and the securing element 130 in resurfacing the corneal surface 105, as described in greater detail herein. As noted above, the eye 104, the corneal surface 105, the corneal epithelium 107, the tear film 109, the eyelid 111, and the one or more surface irregularities 113 comprise the anatomical environment upon which the ocular surface resurfacing system 100 operates, and the ocular surface resurfacing system 100 may be configured to act upon the corneal surface 105.

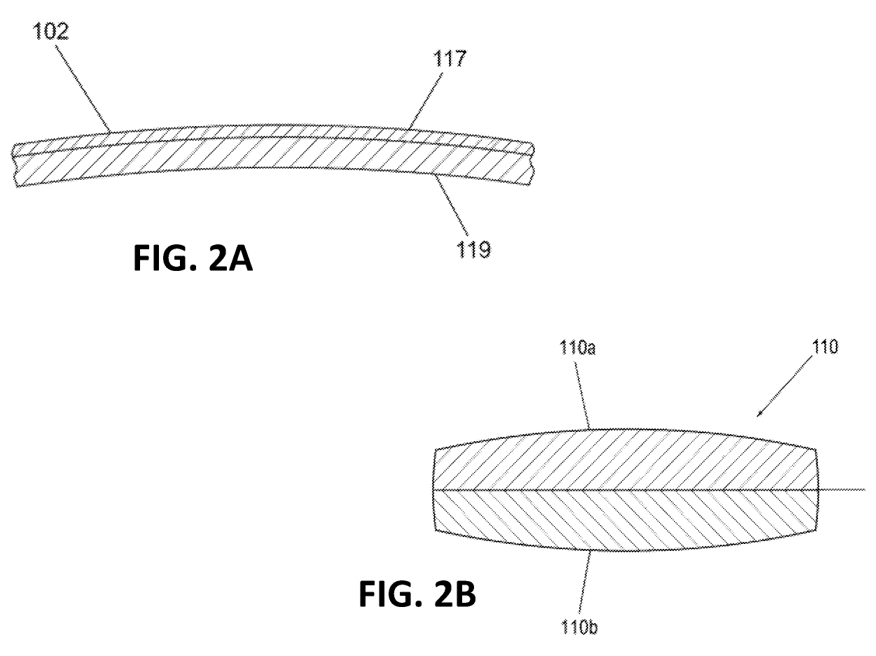

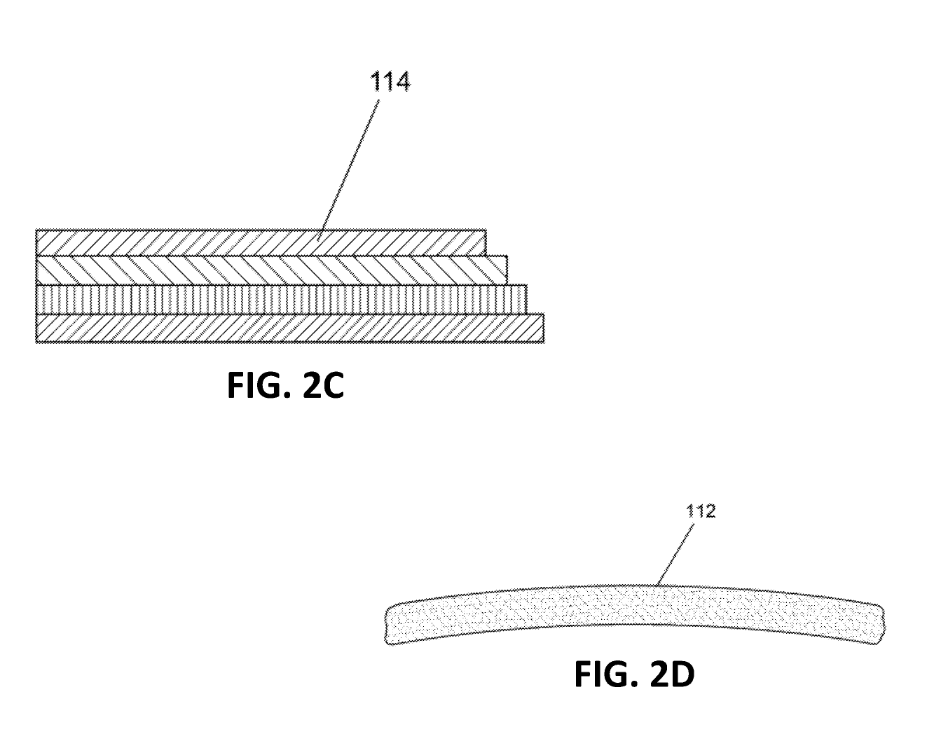

FIGS. 2A–2D show cross-sectional views of various exemplary forms of the amniotic tissue body 102 of the ocular surface resurfacing system 100 implemented in accordance with embodiments of the present disclosure. As shown in FIGS. 2A–2D, the amniotic tissue body 102 may be provided in a handed form having distinguishable faces, as a bilaterally symmetric laminate 110, as a multilayer stack 114, and/or as a homogenized and/or reconstituted non-handed sheet 112, each of which is described in greater detail below.

In embodiments, the amniotic tissue body 102 may be configured to operate as a pliable sheet or body of amniotic tissue configured to be laid onto the corneal surface 105 and to conform to and fill the one or more surface irregularities 113 of the corneal surface 105 so as to present a smooth ocular surface. In embodiments, the amniotic tissue body 102 may be configured to be laid onto the corneal surface 105 without being sutured to the eye 104. By conforming to and filling the one or more surface irregularities 113, the amniotic tissue body 102 may be configured to operate as the resurfacing element of the ocular surface resurfacing system 100.

In embodiments, the amniotic tissue body 102 may be provided in any of a plurality of tissue forms. For example, in embodiments, the amniotic tissue body 102 may comprise a cryopreserved amniotic tissue, including a cryopreserved amniotic tissue provided in a ring form, a dehydrated amniotic tissue, including a dehydrated amniotic tissue provided as a sheet and/or as a powder, and/or a hydrated amniotic tissue. In embodiments, the amniotic tissue body 102 may be provided as an air-dried, non-site-specific starting stock from which the amniotic tissue body 102 may be cut to fit the corneal surface 105, and, in embodiments, a layer of a source amniotic tissue may be separated and/or excluded from the amniotic tissue body 102. In embodiments, the amniotic tissue body 102 may be provided in a sterile condition suitable for application to the corneal surface 105.

In embodiments, as shown in FIG. 2A, the amniotic tissue body 102 may be provided as a handed body having a basement-membrane and/or epithelial-supporting face 117 and a stromal face 119, the basement-membrane and/or epithelial-supporting face 117 being distinguishable from the stromal face 119. In embodiments, the handed body may be configured for an orientation-controlled application in which the basement-membrane and/or epithelial-supporting face 117 is oriented toward the corneal surface 105. It is noted that the handed body of FIG. 2A represents an orientation-controlled embodiment of the amniotic tissue body 102, and that orientation-independent embodiments of the amniotic tissue body 102 are described herein as well.

In embodiments, as shown in FIG. 2B, the amniotic tissue body 102 may be configured as a bilaterally symmetric laminate 110 having a first face 110a and a second face 110b, the first face 110a and the second face 110b presenting equivalent amniotic tissue such that either of the first face 110aand/or the second face 110b may be placed in contact with the corneal surface 105. In embodiments, the bilaterally symmetric laminate 110 may be formed by bonding two layers of amniotic tissue at a central interface, with the two layers oriented such that an outwardly-presenting surface of each layer cooperates to define the equivalent first face 110a and second face 110b of the bilaterally symmetric laminate 110. In this manner, the bilaterally symmetric laminate 110 may be configured to deliver the epithelial-supporting, regenerative, and/or anti-inflammatory functionality of the amniotic tissue body 102 to the corneal surface 105 independent of which of the first face 110a and the second face 110b is placed against the corneal surface 105, such that the amniotic tissue body 102 may be laid onto the corneal surface 105 without orienting a particular face of the amniotic tissue body 102 toward the cornea 105. By configuring the first face 110aand the second face 110b to present equivalent tissue, the bilaterally symmetric laminate 110 of embodiments may be made and/or configured to not include a handedness indicator and may be supplied without requiring it to be marked for a particular face-down orientation.

In embodiments, the orientation-independent functionality described with reference to the bilaterally symmetric laminate 110 may likewise be provided by other non-handed constructions of the amniotic tissue body 102 described herein, including a homogenized and/or reconstituted non-handed sheet 112, a suspension form, and a dropper-formed in-situ membrane, each of which may be configured to present equivalent tissue to the corneal surface 105 regardless of placement orientation. It is noted that the foregoing constructions and methods of forming the amniotic tissue body 102 should not be construed as limiting in any way.

In embodiments, and as shown in FIG. 2D, the amniotic tissue body 102 may be configured as a homogenized and/or reconstituted non-handed sheet 112. In embodiments, the homogenized and/or reconstituted non-handed sheet 112 may comprise an amniotic tissue that has been homogenized and/or reconstituted such that the homogenized and/or reconstituted non-handed sheet 112 lacks a layered handedness. In this manner, a first surface and/or a second surface of the homogenized and/or reconstituted non-handed sheet 112 present equivalent tissue to the corneal surface 105. By configuring the homogenized and/or reconstituted non-handed sheet 112 to lack or forgo a layered handedness, the amniotic tissue body 102 may be configured to be laid onto the corneal surface 105 independent of which surface of the homogenized and/or reconstituted non-handed sheet 112 is placed against the corneal surface 105.

In embodiments, the orientation-independent constructions of the amniotic tissue body 102 may be formed by any of a plurality of techniques. For example, in embodiments, the bilaterally symmetric laminate 110 may be formed by laminating two layers of amniotic tissue such that an outwardly-presenting surface of each layer defines an equivalent outer face of the bilaterally symmetric laminate 110, and the homogenized and/or reconstituted non-handed sheet 112 may be formed by homogenizing and/or reconstituting an amniotic tissue into a sheet lacking a layered handedness. In embodiments, a suspension form of the amniotic tissue body 102 may be formed by suspending an amniotic tissue in a solution, and a dropper-formed in-situ membrane 116 of the amniotic tissue body 102 may be formed by applying a dropper formulation to the corneal surface 105 such that the dropper formulation forms a membrane upon the corneal surface 105, each of the suspension form and the dropper-formed in-situ membrane 116 being inherently non-handed. It is noted that the foregoing techniques are described herein as examples of hot to make and use the orientation-independent constructions of the amniotic tissue body 102 in any of the disclosed forms, and as such, should not be construed as limiting in any way.

In embodiments, and as shown in FIG. 2C, the amniotic tissue body 102 may be configured as a multilayer stack 114 comprising two or more bodies of amniotic tissue. In embodiments, the two or more bodies of the multilayer stack 114 may comprise amniotic tissues of a common tissue type and/or amniotic tissues of two or more different tissue types. In embodiments, the multilayer stack 114 may be configured to provide a selected coverage and/or a selected thickness of amniotic tissue at the corneal surface 105.

In embodiments, the amniotic tissue body 102 may be sized and/or shaped to the corneal surface 105. For example, the amniotic tissue body 102 may be cut to fit the corneal surface 105 from a non-site-specific starting stock, and in some embodiments may be sized to cover a worst-irregularity region of the corneal surface 105, a cataract-relevant region of the corneal surface 105, and/or a standard portion or percentage of the corneal surface 105. In embodiments, the amniotic tissue body 102 may be cut to a contour derived from a corneal topography and/or an irregularity map of the corneal surface 105. In embodiments, the amniotic tissue body 102 may present a non-handed plan-view outline lacking a handedness indicator, and may be provided in a selected thickness and/or as a family of sizes supplied as a kit, as described in greater detail herein. It is noted that any percentage of corneal surface coverage, any number of layers, and any thickness referenced herein are exemplary and should not be construed as limiting in any way.

In embodiments, the amniotic tissue body 102 may be configured to conform to and fill the one or more surface irregularities 113 of the corneal surface 105 so as to present a smooth ocular surface, and to deliver regenerative and/or anti-inflammatory factors of the amniotic tissue body 102 to the corneal surface 105 over a dwell period, the dwell period being, in one non-limiting example, on the order of approximately less than one day, between one and two days and/or less than two days, between two and three days and/or less than three days, between three and four days and/or less than four days, between four and five days and/or less than five days, between five and six days and/or less than six days, between six and seven days and/or less than seven days, between seven and eight days and/or less than eight days, between eight and nine days and/or less than nine days, between nine and ten days and/or less than ten days, between ten and eleven days and/or less than eleven days, between eleven and twelve days and/or less than twelve days, between twelve and thirteen days and/or less than thirteen days, between thirteen and fourteen days and/or less than fourteen days, and in some cases greater than fourteen days. In embodiments, the amniotic tissue body 102 may be configured to be incorporated and/or resorbed by the corneal surface 105 over the dwell period.

In embodiments, the amniotic tissue body 102 may be configured to interoperate with the corneal surface 105 and with other components of the ocular surface resurfacing system 100. For example, in embodiments, the amniotic tissue body 102 may be configured to be placed in direct conforming contact with the corneal surface 105, to be retained against the corneal surface 105 by the securing element 130, including by being retained beneath and/or enclosed within the securing element 130, to be retained against the corneal surface 105 by way of a developed-tackiness interface 136 of the corneal surface 105, as described in greater detail herein, and/or to be smoothed against the corneal surface 105 by a sponge 150, as described in greater detail herein.

In embodiments, the amniotic tissue body 102 may be provided in any of a plurality of variations. For example, the amniotic tissue body 102 may be provided as a cryopreserved, a dehydrated, and/or a hydrated amniotic tissue, may be provided as a single-layer body and/or as a multilayer stack 114, may be provided as an orientation-controlled handed body and/or as an orientation-independent construction, and/or may be provided as a body that has been subjected to a controlled rehydration with a defined solution to tune a conformability of the amniotic tissue body 102 and/or to a thermal conditioning configured to improve a conformance of the amniotic tissue body 102 to the corneal surface 105. It is noted that, in embodiments, the terms “amniotic tissue” and “amniotic membrane” may include cryopreserved, dehydrated, and hydrated amniotic tissues, as well as hybrid assemblies incorporating an amniotic tissue, as described in greater detail herein.

In embodiments, the functionality of the amniotic tissue body 102 to conform to and fill the one or more surface irregularities 113 of the corneal surface 105 may be provided by a pliable sheet or body of amniotic tissue provided in a cryopreserved, dehydrated, and/or hydrated form, and may be further provided by a controlled rehydration and/or a thermal conditioning configured to tune a conformability of the amniotic tissue body 102, and by a selected thickness of the amniotic tissue body 102. In embodiments, the functionality of the amniotic tissue body 102 to function independent of which face contacts the corneal surface 105 may be provided by the bilaterally symmetric laminate 110, the homogenized and/or reconstituted non-handed sheet 112, the inherently non-handed suspension form, and/or the inherently non-handed dropper-formed in-situ membrane 116. It is noted that the foregoing structures are exemplary of the structures that may provide the described functionalities of the amniotic tissue body 102, and that equivalent structures providing the same functionalities are contemplated and should not be construed as limited to the disclosed structures in any way.

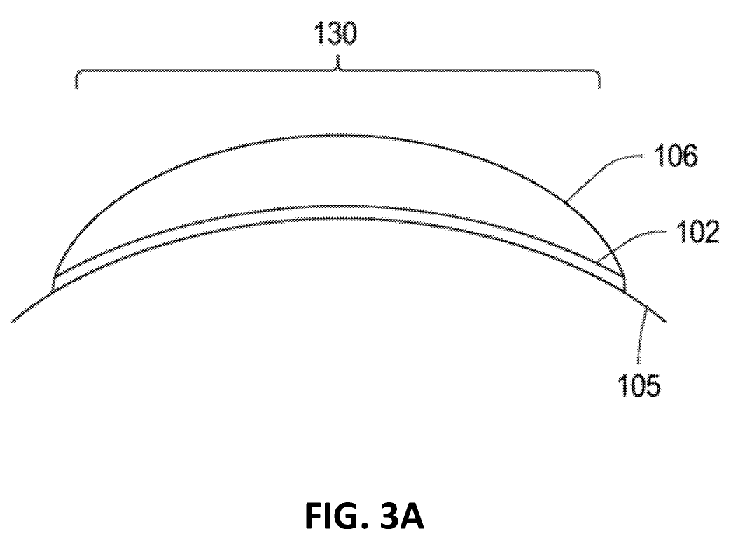

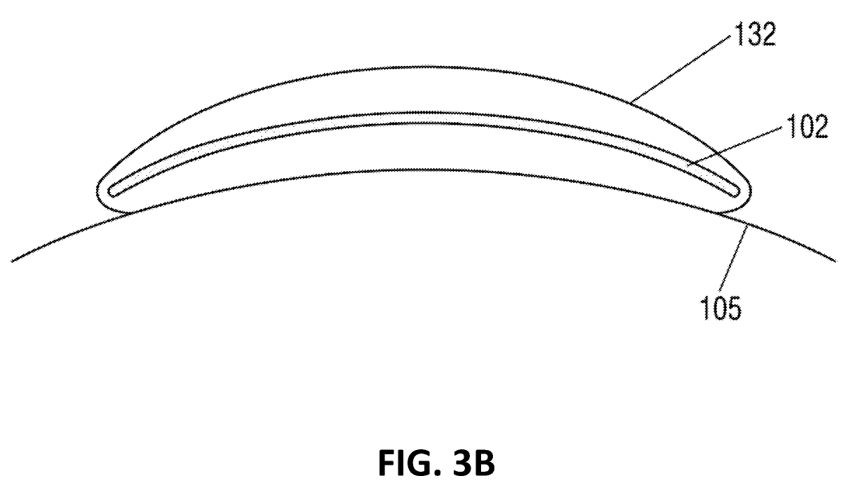

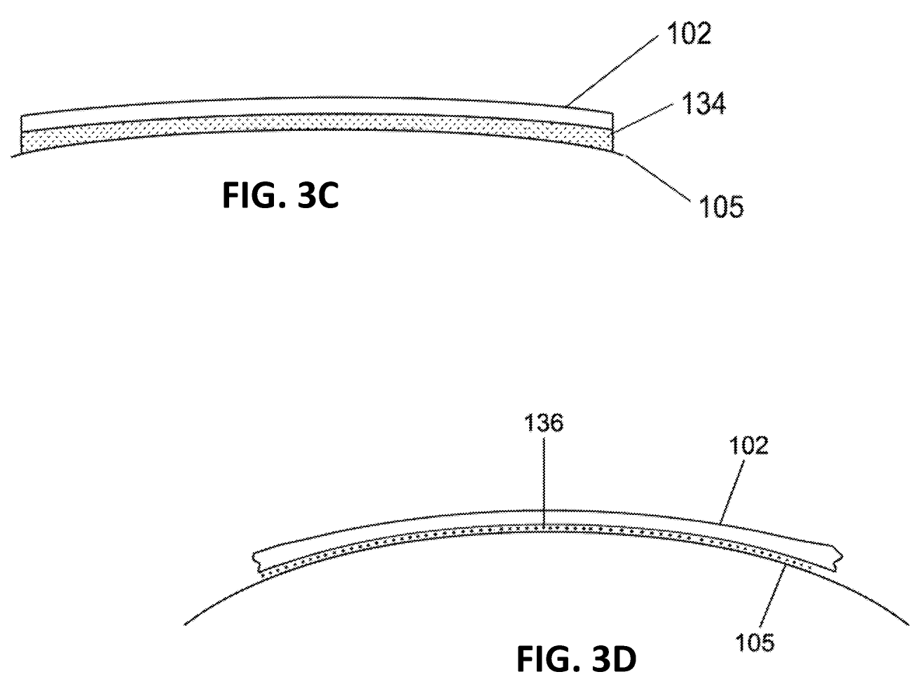

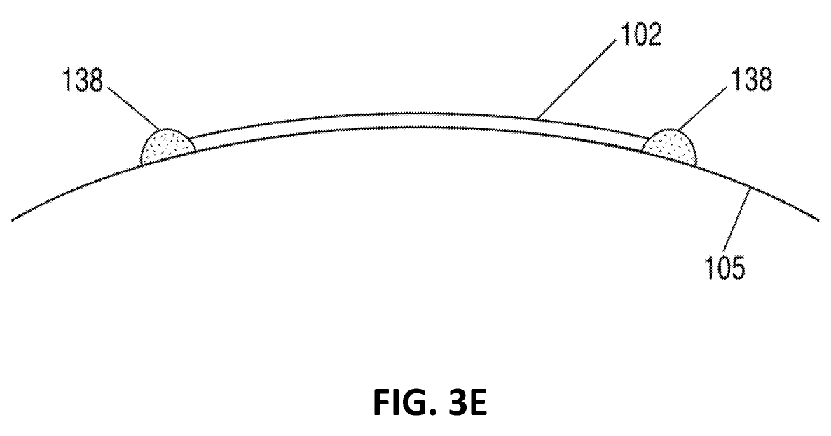

FIGS. 3A-3E show cross-sectional views, taken at an interface between the amniotic tissue body 102 and the corneal surface 105, of various examples of the securing element 130 of the ocular surface resurfacing system 100 implemented in accordance with embodiments of the present disclosure. As shown in FIGS. 3A-3E, the securing element 130 may be instantiated or implemented as a bandage contact lens 106, an enclosing assembly 132, an applied adhesive interface 134, a native developed-tackiness interface 136, and/or a fibrin interface 138, each of which is described in greater detail below.

In embodiments, the securing element 130 may be configured to operate as a component element configured to retain the amniotic tissue body 102 against the corneal surface 105. In embodiments, the securing element 130 may be configured to retain the amniotic tissue body 102 against the corneal surface 105 without the amniotic tissue body 102 being sutured to the eye 104. By retaining the amniotic tissue body 102 against the corneal surface 105, the securing element 130 may be configured to maintain the amniotic tissue body 102 in a conforming relationship with the one or more surface irregularities 113 of the corneal surface 105 over a dwell period. It is noted that in some embodiments, the securing element 130 may be optional and the ocular surface resurfacing system 100 may forego the securing element 130 and in these embodiments, the amniotic tissue body 102 may not be retained against the corneal surface 105 and may instead be allowed to move or dislodge from the corneal surface 105.

In embodiments, and as shown in FIG. 3A, the securing element 130 may be instantiated or implemented as a bandage contact lens 106 configured to be placed over the amniotic tissue body 102 such that the amniotic tissue body 102 is retained between the bandage contact lens 106 and the corneal surface 105. In embodiments, the bandage contact lens 106 may be configured to retain and to protect the amniotic tissue body 102 against the corneal surface 105 over the dwell period.

In embodiments, and as shown in FIG. 3B, the securing element 130 may be instantiated or implemented as an enclosing assembly 132 in which the amniotic tissue body 102 is held within the bandage contact lens 106, such that the amniotic tissue body 102 and the bandage contact lens 106 may be placed together upon the corneal surface 105 as a hybrid lens-membrane assembly. In embodiments, the enclosing assembly 132 may be configured to retain the amniotic tissue body 102 within the bandage contact lens 106 prior to and during application to the corneal surface 105.

In embodiments, and as shown in FIG. 3C, the securing element 130 may be instantiated or implemented as an applied adhesive interface 134 disposed on the amniotic tissue body 102 and/or on the corneal surface 105. In embodiments, the applied adhesive interface 134 may be configured to retain the amniotic tissue body 102 against the corneal surface 105, and may be configured to accommodate a variance in a curvature between the amniotic tissue body 102 and the corneal surface 105.

In embodiments, and as shown in FIG. 3D, the securing element 130 may be instantiated or implemented as a native developed-tackiness interface 136, by which the amniotic tissue body 102 is retained against the corneal surface 105 without a separate securing article. As described herein, in embodiments, the native developed-tackiness interface 136 may be formed by removing moisture from the corneal surface 105, including by wicking moisture from the corneal surface 105 (e.g., with a sponge 150) over a period of time (e.g., anywhere between 20-120 seconds, such as approximately between 15 and 25 seconds, approximately between 25 and 35 seconds, approximately between 35 and 45 seconds, approximately between 45 and 55 seconds, approximately between 55 and 65 seconds, approximately between 65 and 75 seconds, approximately between 75 and 85 seconds, approximately between 85 and 95 seconds, approximately between 95 and 105 seconds, approximately between 105 and 115 seconds, approximately between 115 and 125 seconds, and/or in some cases greater than 125 seconds), such that the corneal epithelium 107 develops a tacky consistency by which the amniotic tissue body 102 is retained against the corneal surface 105. By forming the native developed-tackiness interface 136 through a removal of surface moisture from the corneal surface 105, the ocular surface resurfacing system 100 may be configured to retain the amniotic tissue body 102 against the corneal surface 105 by way of a dry adhesion, as distinct from an application of a saline and/or a rehydrating solution to the corneal surface 105. It is noted that, in embodiments, the native developed-tackiness interface 136 may be formed without rehydrating the corneal surface 105 with a saline, and that the developed tackiness of the corneal epithelium 107 is a response of the corneal surface 105 to the removal of surface moisture by the ocular surface resurfacing system 100.

In embodiments, and as shown in FIG. 3E, the securing element 130 may be instantiated as a fibrin interface 138 comprising a fibrin and/or a tissue sealant disposed between the amniotic tissue body 102 and the corneal surface 105. In embodiments, the fibrin interface 138 may be configured to retain the amniotic tissue body 102 against the corneal surface 105 over the dwell period.

In embodiments, the securing element 130 may be configured to hold the amniotic tissue body 102 in position and/or centered upon the corneal surface 105, and/or to protect the amniotic tissue body 102 and the corneal surface 105 over the dwell period. In embodiments, the securing element 130 may be configured to interoperate with the amniotic tissue body 102, with the corneal surface 105 and the corneal epithelium 107, with the sponge 150 by which the amniotic tissue body 102 may be smoothed against the corneal surface 105 prior to securing, and/or with an eyelid speculum 160 by which the eye 104 may be held open during placement, as described in greater detail herein. In embodiments, the securing element 130 may be applied with or without a bandage contact lens 106 depending upon a clinical need.

In embodiments, the functionality of the securing element 130 to retain the amniotic tissue body 102 against the corneal surface 105 may be provided by the bandage contact lens 106 placed over the amniotic tissue body 102, the enclosing assembly 132 holding the amniotic tissue body 102 within the bandage contact lens 106, the applied adhesive interface 134, the native developed-tackiness interface 136 formed by removing surface moisture from the corneal surface 105, the fibrin interface 138, and/or a natural adherence of the amniotic tissue body 102 to the corneal surface 105. It is noted that the foregoing structures are exemplary of the structures that may provide the retention functionality of the securing element 130, and that equivalent structures providing the same functionality are contemplated and should not be construed as limited to the disclosed structures in any way.

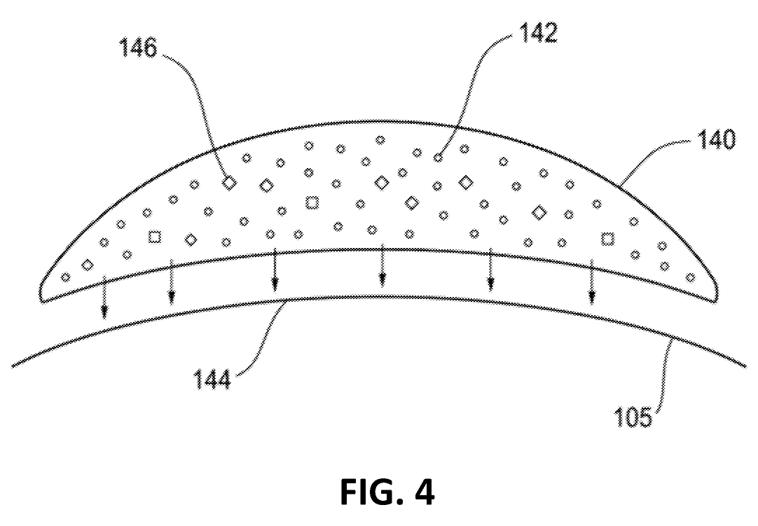

FIG. 4 shows a cross-sectional view of a therapeutic-eluting lens 140 of the ocular surface resurfacing system 100 implemented in accordance with embodiments of the present disclosure. In embodiments, the therapeutic-eluting lens 140 may be configured to operate as an example implementation of the securing element 130 that is configured to operate as an active delivery vehicle, such that the therapeutic-eluting lens 140 may be configured to retain the amniotic tissue body 102 against the corneal surface 105 and/or to deliver one or more therapeutic factors to the corneal surface 105.

In embodiments, the therapeutic-eluting lens 140 may comprise one or more amniotic stem cells 142 embedded within a matrix of the therapeutic-eluting lens 140, an amniotic material 144 absorbed into the therapeutic-eluting lens 140 from a soaking solution, and/or one or more adjunct loads 146. In embodiments, the one or more adjunct loads 146 may include a hyaluronic acid, an autologous serum and/or a platelet-rich plasma, an anti-inflammatory agent and/or a steroid, a preservative-free formulation, etc.

In embodiments, the therapeutic-eluting lens 140 may be configured to release the one or more amniotic stem cells 142, the amniotic material 144, and/or the one or more adjunct loads 146 to the corneal surface 105 over a dwell period. By releasing the one or more therapeutic factors to the corneal surface 105 over the dwell period, the therapeutic-eluting lens 140 may be configured to operate as an active delivery vehicle, as distinct from a passive cover over the corneal surface 105.

In embodiments, the therapeutic-eluting lens 140 may be configured to interoperate with the corneal surface 105, with the soaking solution 184 from which the amniotic material 144 may be absorbed into the therapeutic-eluting lens 140, and/or with a follow-up process described in greater detail herein. In embodiments, the therapeutic-eluting lens 140 may be provided as a stem-cell-eluting lens, a solution-loaded lens, and/or an adjunct-loaded lens, and may be combined with a separate amniotic tissue body 102 and/or used alone upon the corneal surface 105. It is noted that the foregoing structures, factors, and variations of the therapeutic-eluting lens 140 are exemplary and should not be construed as limiting in any way.

In embodiments, the ocular surface resurfacing system 100 may include one or more instruments and/or one or more agents configured to cooperate with the amniotic tissue body 102 and the securing element 130 in resurfacing the corneal surface 105. In embodiments, the one or more instruments and/or the one or more agents may comprise a sponge 150, an eyelid speculum 160, a dropper 170, a syringe 180, an anesthetic agent 190, and/or a punctal plug (not shown), each of which is described in greater detail herein and each of which may be depicted in connection with an operative sequence described herein.

In embodiments, the sponge 150 may be configured to wick moisture from the corneal surface 105 so as to develop a tacky consistency of the corneal epithelium 107, as described with reference to the native developed-tackiness interface 136, and/or to smooth and even the amniotic tissue body 102 against the corneal surface 105. In embodiments, the sponge 150 may be provided as an anesthetic-loaded sponge 152 configured to work an anesthetic into the amniotic tissue body 102 and/or the corneal surface 105 during application.

In embodiments, the eyelid speculum 160 may be configured to retract and/or hold the eyelid 111 so as to hold the eye 104 open during application of the amniotic tissue body 102 to the corneal surface 105. In embodiments, the eyelid speculum 160 may be configured to aid placement of the amniotic tissue body 102 upon the corneal surface 105, including where a blinking of the eye 104 may otherwise impede placement.

In embodiments, the dropper 170 may be configured to deliver a formulation to the corneal surface 105 such that the formulation forms an in-situ membrane 116 upon the corneal surface 105, as described in greater detail herein. In embodiments, the in-situ membrane 116 formed by the dropper 170 may be configured to operate as a form of the amniotic tissue body 102 that is inherently non-handed.

In embodiments, the syringe 180 may be configured to deliver an amniotic suspension formulation 182 to the corneal surface 105 for a direct ocular-surface application of the amniotic suspension formulation 182, as described in more detail herein. In embodiments, the amniotic suspension formulation 182 may be applied to the corneal surface 105 as a supplement to a cut graft of the amniotic tissue body 102 and/or in lieu of a cut graft of the amniotic tissue body 102. In embodiments, the ocular surface resurfacing system 100 may comprise a contact-lens soaking solution 184 comprising an amniotic material, the contact-lens soaking solution 184 being configured to load the therapeutic-eluting lens 140 with the amniotic material 144 by absorption.

In embodiments, the anesthetic agent 190 may be configured to be applied to the eye 104 so as to provide patient comfort during application of the amniotic tissue body 102 to the corneal surface 105. In embodiments, the anesthetic agent 190 may be applied as a topical agent and/or by way of the anesthetic-loaded sponge 152.

In embodiments, the punctal plug may be configured to be placed in a punctum of the eye 104 so as to provide an adjunct retention of a tear film 109 and/or of one or more therapeutic factors at the corneal surface 105 over a dwell period.

In embodiments, the one or more instruments and/or the one or more agents may be provided in any of a plurality of exemplary variations. For example, in embodiments, the sponge 150 may be provided as an anesthetic-loaded sponge 152, the amniotic tissue body 102 may be applied with and/or without the eyelid speculum 160 depending upon a clinical need, and the amniotic suspension formulation 182 may be applied by the syringe 180 and/or formed in situ by the dropper 170. It is noted that the foregoing instruments, agents, and variations are exemplary and should not be construed as limiting in any way.

In embodiments, the ocular surface resurfacing system 100 may include one or more diagnostic adjuncts configured to inform a diagnosis, a readiness, a sizing, and/or a follow-up associated with resurfacing the corneal surface 105. For example, in embodiments, the one or more diagnostic adjuncts may comprise a corneal imaging and/or topography device 192 configured to obtain a corneal topography and/or an irregularity map of the corneal surface 105, and/or a biometric measurement device configured to obtain a biometric measurement of the eye 104.

In embodiments, the one or more diagnostic adjuncts may further comprise a sodium fluorescein staining configured to inform an assessment of a re-epithelialization of the corneal surface 105, a photographic and/or microscopic documentation configured to inform an assessment of a centering and/or a healing of the amniotic tissue body 102 at the corneal surface 105, a patient questionnaire 302, and/or a clinical examination 304.

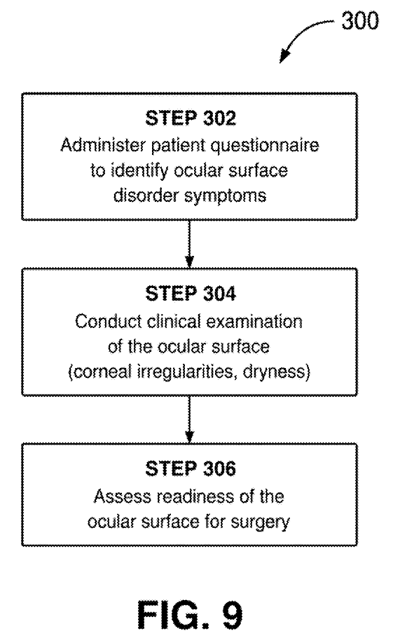

In embodiments, the one or more diagnostic adjuncts may be configured to interoperate with one or more processes described with reference to FIG. 9 through FIG. 15 herein, including to inform a diagnosis of a condition of the corneal surface 105, a readiness of the corneal surface 105 for a biometric measurement and/or an ocular surgery, a sizing of the amniotic tissue body 102, and/or a follow-up assessment of the corneal surface 105 over a dwell period. It is noted that the foregoing diagnostic adjuncts and their functionalities are exemplary and should not be construed as limiting in any way.

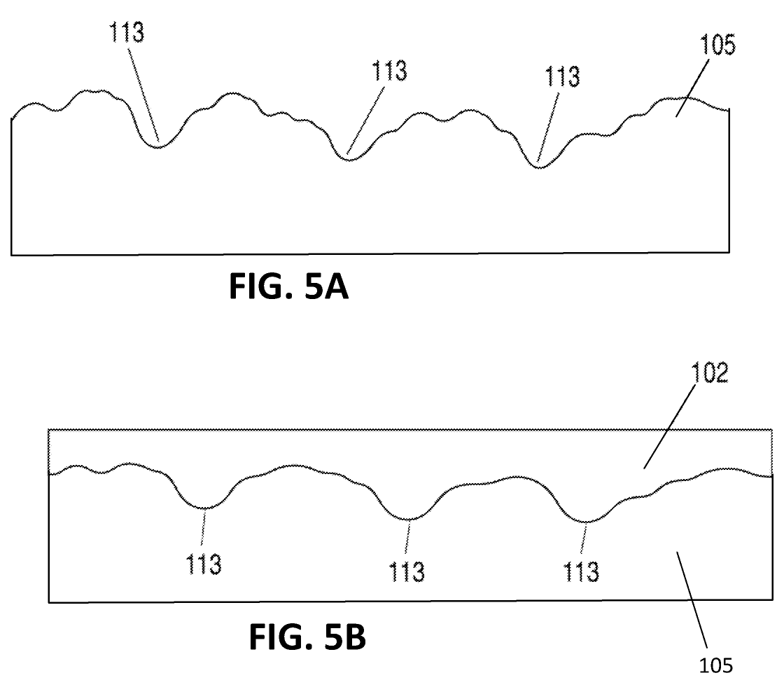

FIGS. 5A and 5B show an example of a magnified surface profiles of the corneal surface 105 of the eye 104 before and after resurfacing by the ocular surface resurfacing system 100 in accordance with embodiments of the present disclosure. For example, FIG. 5A shows the corneal surface 105 presenting one or more surface irregularities 113 prior to resurfacing, and FIG. 5B shows the amniotic tissue body 102 conformed over the corneal surface 105 so as to fill the one or more surface irregularities 113 and to present a smooth ocular surface.

In embodiments, as shown in FIG. 5A, the corneal surface 105 may present one or more surface irregularities 113, the one or more surface irregularities 113 comprising one or more depressions, elevations, and/or other deviations of the corneal surface 105 from a smooth ocular surface. In embodiments, the one or more surface irregularities 113 may distort an ocular surface presented by the corneal surface 105, such that a wavefront and/or a reflection at the corneal surface 105 is perturbed by the one or more surface irregularities 113.

In embodiments, the one or more surface irregularities 113 of the corneal surface 105 may propagate into an error in a measurement obtained from the corneal surface 105. For example, in embodiments, the one or more surface irregularities 113 may introduce an error into a corneal topography and/or an irregularity map obtained from the corneal surface 105, into a biometric measurement obtained from the eye 104, and/or into an intraocular lens calculation derived from the biometric measurement, such that the one or more surface irregularities 113 may propagate into a topographic error, a biometric error, and/or an intraocular lens selection error. The ocular surface resurfacing system 100 recognizes that the one or more surface irregularities 113 of the corneal surface 105 may propagate into such errors, and the ocular surface resurfacing system 100 is configured to address this by conforming the amniotic tissue body 102 over the corneal surface 105 so as to fill the one or more surface irregularities 113 and to present a smooth ocular surface from which a measurement may be obtained.

In embodiments, as shown in FIG. 5B, the amniotic tissue body 102 may be laid onto the corneal surface 105 such that the amniotic tissue body 102 conforms to and fills the one or more surface irregularities 113 of the corneal surface 105. In embodiments, the amniotic tissue body 102 may be configured to fill the one or more surface irregularities 113 in a manner of filling one or more potholes of the corneal surface 105, such that an outwardly-presenting surface 199 of the amniotic tissue body 102 presents a smooth and even ocular surface over the corneal surface 105. By filling the one or more surface irregularities 113 to present a smooth and even ocular surface, the amniotic tissue body 102 may be configured to operate as a resurfacing layer over the corneal surface 105.

In embodiments, the smooth and even ocular surface (e.g., the outwardly-presenting surface 199) presented by the amniotic tissue body 102 over the corneal surface 105 may be configured to reduce an error that the one or more surface irregularities 113 may otherwise propagate into a measurement obtained from the corneal surface 105. For example, in embodiments, by presenting a smooth and even ocular surface, the amniotic tissue body 102 may be configured to improve an accuracy of a corneal topography and/or a corneal mapping of the corneal surface 105, of a biometric measurement obtained from the eye 104, and/or of an intraocular lens calculation derived from the biometric measurement, prior to an ocular surgery.

In embodiments, the ocular surface resurfacing system 100 may be configured to resurface the corneal surface 105 for the object of presenting a smooth ocular surface from which a measurement may be obtained, as distinct from a use of the amniotic tissue body 102 as a passive patch over a diseased corneal surface 105. By resurfacing the corneal surface 105 to present a smooth ocular surface for a measurement, the ocular surface resurfacing system 100 may be configured to optimize the corneal surface 105 for a biometric and/or an ocular measurement prior to an ocular surgery. It is noted that the foregoing description of the resurfacing of the corneal surface 105 and the measurements associated therewith is exemplary and should not be construed as limiting in any way.

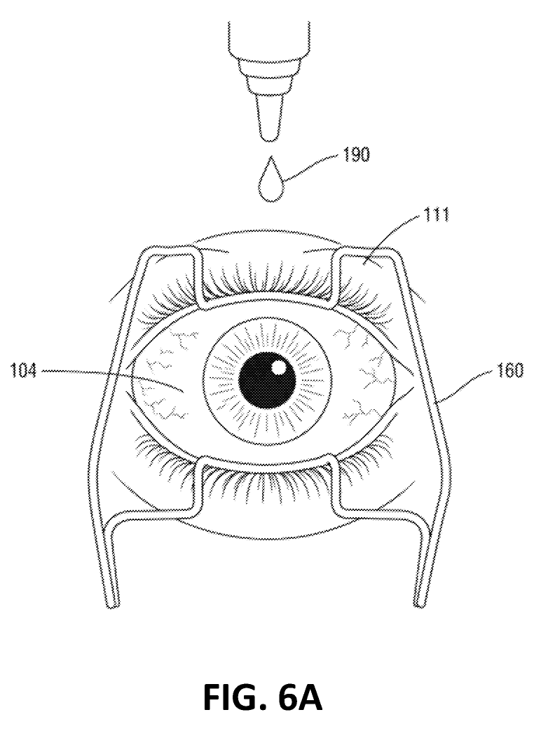

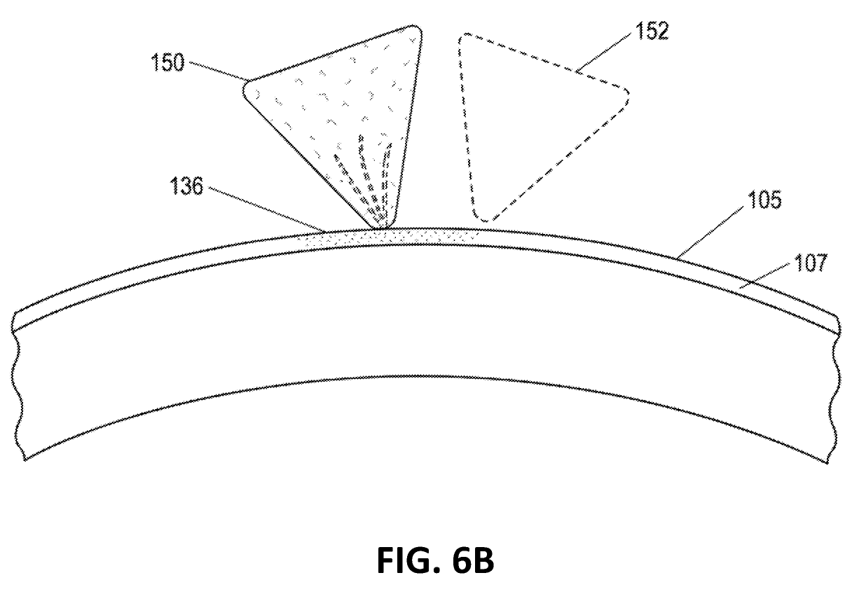

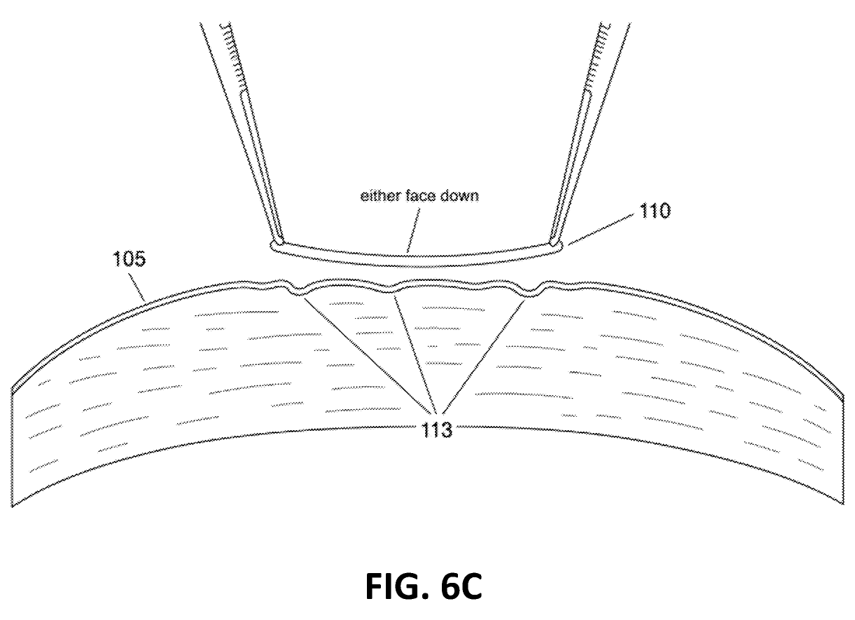

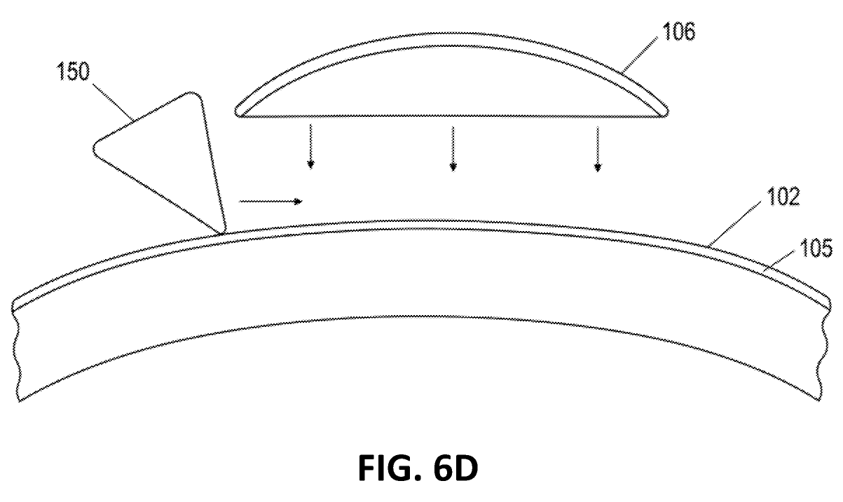

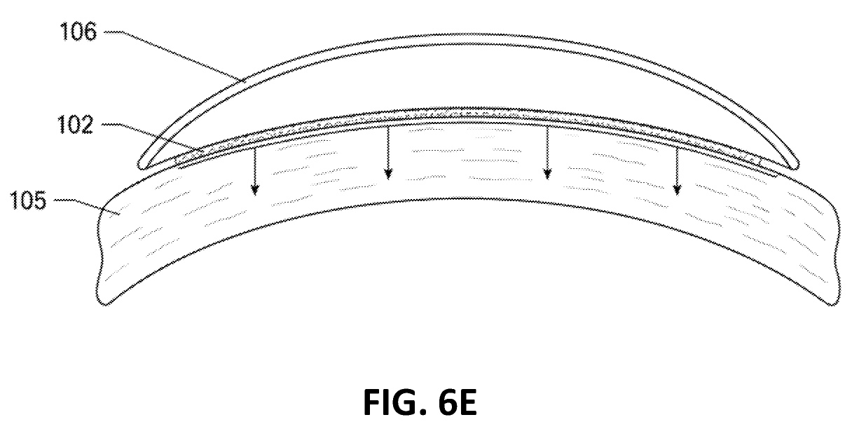

FIGS. 6A-6E show the ocular surface resurfacing system 100 at successive stages of operation upon the corneal surface 105 of the eye 104 in accordance with embodiments of the present disclosure. As shown in FIGS. 6A-6E, the corneal surface 105 may be prepared and the amniotic tissue body 102 may be laid onto, conformed to, smoothed against, secured to, and allowed to dwell upon the corneal surface 105, as described in greater detail below.

In embodiments, as shown in FIG. 6A, an anesthetic agent 190 may be applied to the eye 104, and the eyelid 111 may be retracted by an eyelid speculum 160 so as to hold the eye 104 open during the application. In embodiments, the eye 104 may be held open by the eyelid speculum 160 for an interval sufficient to perform the application, the interval being, in one non-limiting example, on the order of approximately anywhere between 20-120 seconds, such as approximately between 15 and 25 seconds, approximately between 25 and 35 seconds, approximately between 35 and 45 seconds, approximately between 45 and 55 seconds, approximately between 55 and 65 seconds, approximately between 65 and 75 seconds, approximately between 75 and 85 seconds, approximately between 85 and 95 seconds, approximately between 95 and 105 seconds, approximately between 105 and 115 seconds, approximately between 115 and 125 seconds, and/or in some cases greater than 125 seconds.

In embodiments, as shown in FIG. 6B, moisture may be wicked from the corneal surface 105 with a sponge 150 and/or an anesthetic-loaded sponge 152 so as to develop a native developed-tackiness interface 136 at the corneal epithelium 107. In embodiments, the corneal epithelium 107 develops a tacky consistency in response to the removal of moisture from the corneal surface 105 by the sponge 150 over the period of time (e.g., e.g., anywhere between 20-120 seconds, such as approximately between 15 and 25 seconds, approximately between 25 and 35 seconds, approximately between 35 and 45 seconds, approximately between 45 and 55 seconds, approximately between 55 and 65 seconds, approximately between 65 and 75 seconds, approximately between 75 and 85 seconds, approximately between 85 and 95 seconds, approximately between 95 and 105 seconds, approximately between 105 and 115 seconds, approximately between 115 and 125 seconds, and/or in some cases greater than 125 seconds). By developing the native developed-tackiness interface 136 through a removal of surface moisture over a time, the ocular surface resurfacing system 100 may be configured to prepare the corneal surface 105 to receive the amniotic tissue body 102 by way of a dry adhesion, and the corneal surface 105 may be prepared in this manner without an application of a saline and/or a rehydrating solution to the corneal surface 105.

In embodiments, as shown in FIG. 6C, the amniotic tissue body 102 may be laid onto the corneal surface 105 such that the amniotic tissue body 102 is retained against the corneal surface 105 by the native developed-tackiness interface 136. In embodiments, the amniotic tissue body 102 may be provided as a bilaterally symmetric laminate 110 laid onto the corneal surface 105 with either of the first face 110aand/or the second face 110b contacting the corneal surface 105. In embodiments, the amniotic tissue body 102 conforms to and fills the one or more surface irregularities 113 of the corneal surface 105 so as to present a smooth ocular surface.

In embodiments, the amniotic tissue body 102 may be laid onto the corneal surface 105 without debriding and/or scraping the corneal surface 105 and/or the corneal epithelium 107, such that the corneal epithelium 107 is preserved beneath the amniotic tissue body 102. For example, the amniotic tissue body 102 may be laid onto the corneal surface 105 as a non-site-specific body that is not configured for a particular site of the corneal surface 105. In embodiments, the amniotic tissue body 102 may be laid onto the corneal surface 105 without orienting a stromal side of the amniotic tissue body 102 toward the cornea, as enabled by the bilaterally symmetric laminate 110 and/or other non-handed constructions of the amniotic tissue body 102 described herein. It is noted that the foregoing manner of laying the amniotic tissue body 102 onto the corneal surface 105 without debridement and/or scraping, as a non-site-specific body, and without a required stromal-side orientation, is described as a feature of the application of the amniotic tissue body 102 to the corneal surface 105.

In embodiments, and as shown in FIG. 6D, the amniotic tissue body 102 may be smoothed and evened against the corneal surface 105 with a sponge 150, and may be secured against the corneal surface 105 by a bandage contact lens 106 placed over the amniotic tissue body 102. In embodiments, the amniotic tissue body 102 may be secured against the corneal surface 105 by way of the native developed-tackiness interface 136 and/or an applied adhesive interface 134 in lieu of and/or in addition to the bandage contact lens 106.

In embodiments, as shown in FIG. 6E, the amniotic tissue body 102 may be allowed to dwell upon the corneal surface 105 over a dwell period, during which the amniotic tissue body 102 may deliver regenerative and/or anti-inflammatory factors to the corneal surface 105 and a smooth ocular surface may be established over the corneal surface 105. In embodiments, the dwell period may be, in one non-limiting example, on the order of approximately less than one day, between one and two days and/or less than two days, between two and three days and/or less than three days, between three and four days and/or less than four days, between four and five days and/or less than five days, between five and six days and/or less than six days, between six and seven days and/or less than seven days, between seven and eight days and/or less than eight days, between eight and nine days and/or less than nine days, between nine and ten days and/or less than ten days, between ten and eleven days and/or less than eleven days, between eleven and twelve days and/or less than twelve days, between twelve and thirteen days and/or less than thirteen days, between thirteen and fourteen days and/or less than fourteen days, and in some cases greater than fourteen days.

In embodiments, the anesthetic agent 190 may be applied to the eye 104 in connection with each of the foregoing stages of operation as appropriate for patient comfort, and the amniotic tissue body 102 may be applied to the corneal surface 105 with and/or without a bandage contact lens 106 depending upon a clinical need. It is noted that the intervals, the stages, and the manner of operation described with reference to FIGS. 6A-6E are exemplary, and that the foregoing should not be construed as limiting in any way.

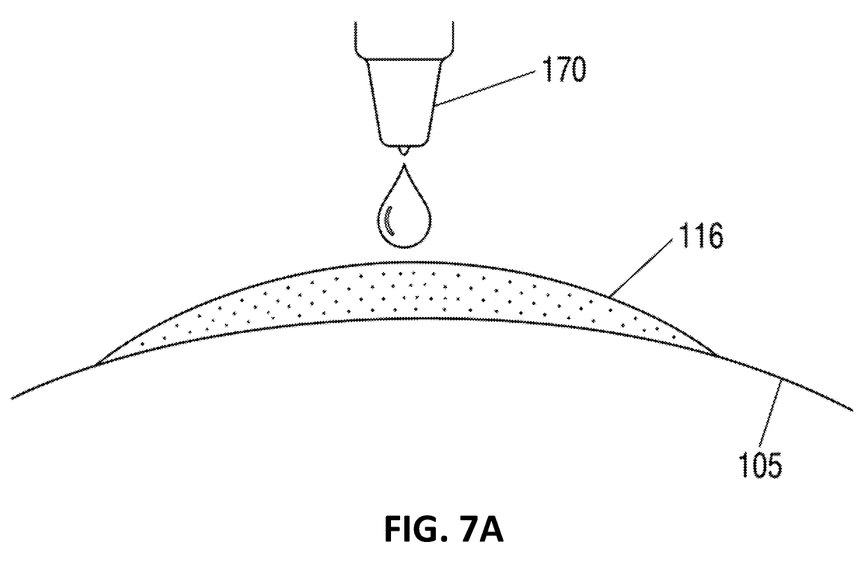

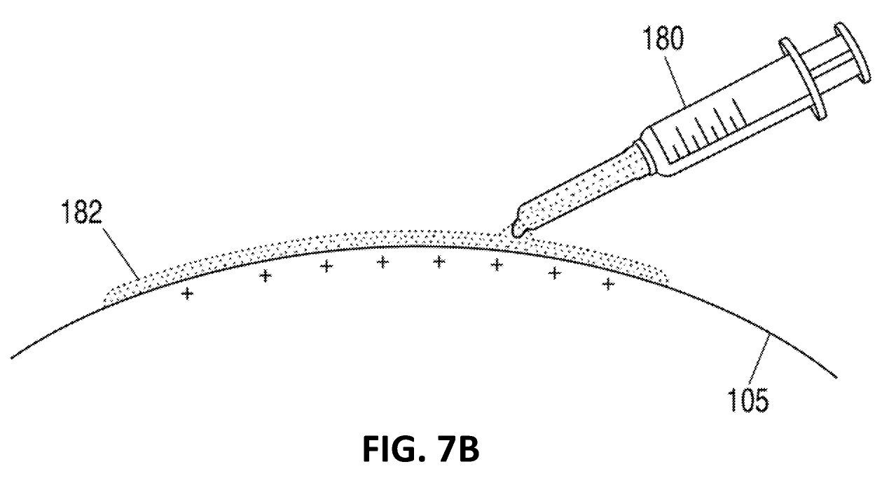

FIGS. 7A and 7B show the ocular surface resurfacing system 100 during alternative modes of delivery of an amniotic tissue to the corneal surface 105 of the eye 104 in accordance with embodiments of the present disclosure. As shown in FIGS. 7A and 7B, an amniotic tissue may be delivered to the corneal surface 105 by way of a dropper 170 that forms an in-situ membrane 116 upon the corneal surface 105, and/or by way of a syringe 180 that applies an amniotic suspension formulation 182 to the corneal surface 105.

In embodiments, as shown in FIG. 7A, a dropper 170 may be configured to dispense a formulation onto the corneal surface 105 such that the formulation forms an in-situ membrane 116 upon the corneal surface 105. In embodiments, the in-situ membrane 116 may be configured to operate as a form of the amniotic tissue body 102 that conforms to and fills the one or more surface irregularities 113 of the corneal surface 105.

In embodiments, the dropper 170 may be configured to dispense the formulation onto the corneal surface 105 as a liquid and/or a flowable formulation that sets, gels, and/or coalesces upon the corneal surface 105 so as to form the in-situ membrane 116 in conformance with the corneal surface 105 and the one or more surface irregularities 113. By forming the in-situ membrane 116 upon the corneal surface 105 from a dispensed formulation, the ocular surface resurfacing system 100 may be configured to present an amniotic tissue body 102 that is inherently non-handed, such that the in-situ membrane 116 presents equivalent tissue to the corneal surface 105 regardless of a placement orientation. In embodiments, the amniotic suspension formulation 182 described with reference to FIG. 7B may likewise be inherently non-handed, such that the in-situ membrane 116 and the amniotic suspension formulation 182 may each be applied to the corneal surface 105 without orienting a particular face toward the cornea.

In embodiments, and as shown in FIG. 7B, a syringe 180 may be configured to apply an amniotic suspension formulation 182 across the corneal surface 105 for a direct ocular-surface application of the amniotic suspension formulation 182. In some additional or alternative embodiments, the corneal surface 105 may be charged so as to attract the amniotic suspension formulation 182 to the corneal surface 105, and/or the amniotic suspension formulation 182 may be taken up at the corneal surface 105 by way of an iontophoresis-assisted uptake, so as to increase a bioavailability of the amniotic suspension formulation 182 at the corneal surface 105.

In embodiments, the amniotic suspension formulation 182 may be formulated using an amniotic tissue base (e.g., a dehydrated amniotic tissue base, a cryopreserved amniotic tissue base, etc.), in which the amniotic tissue base is combined with a wetting agent (e.g., a non-preserved artificial tear compound, saline solution, preserved artificial tear compound, etc.) to generate the amniotic suspension formulation 182.

In embodiments, the alternative modes of delivery described with reference to FIGS. 7A and 7B may be interchangeable with a cut-graft technique described with reference to FIG. 6, and may be applied to the corneal surface 105 in lieu of and/or as a supplement to a cut graft of the amniotic tissue body 102. It is noted that the modes of delivery described herein are exemplary and may be selected depending upon a clinical need, and should not be construed as limiting in any way.

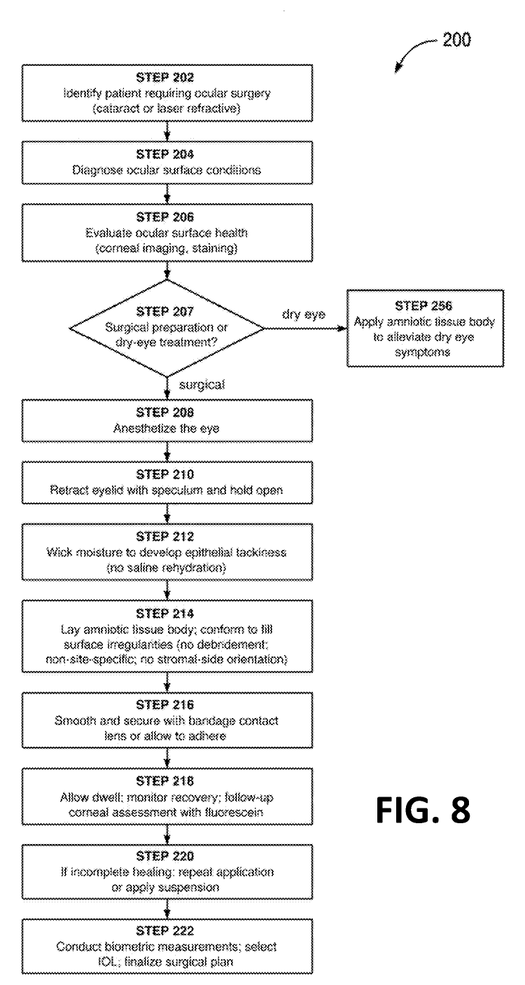

FIG. 8 shows a high-level flow diagram of a method 200 of preparing a corneal surface 105 for an ocular surgery in accordance with embodiments of the present disclosure. In embodiments, the method 200 may be configured to prepare, fix, and/or optimize the corneal surface 105 of the eye 104 by resurfacing the corneal surface 105 with an amniotic tissue body 102 so as to present a smooth ocular surface for a biometric measurement and/or an ocular surgery. It is noted that the method 200 is described herein with reference to the components of the ocular surface resurfacing system 100 and the operative sequences described with reference to FIGS. 1-7, and that the steps of the method 200 may be performed in any suitable order and with one or more steps omitted, as described in greater detail below.

In embodiments, and as shown in FIG. 8, at STEP 202 a patient may be identified, the patient comprising a candidate for an ocular surgery and/or a patient presenting a condition of the corneal surface 105. At STEP 204, one or more conditions of the corneal surface 105 may be diagnosed, including one or more surface irregularities 113 and/or a dryness of the corneal surface 105, and at STEP 206 a health of the corneal surface 105 may be evaluated, including by way of a corneal imaging and/or topography device 192, a biometric measurement device 194, a patient questionnaire 302, and/or a clinical examination 304.

At STEP 207 a determination may be made as to whether the corneal surface 105 is to be prepared for an ocular surgery and/or treated for a dry-eye condition. In embodiments, in response to a determination at STEP 207 that the corneal surface 105 is to be treated for a dry-eye condition, the method 200 may proceed at STEP 256 to a dry-eye treatment method 212, in which the amniotic tissue body 102 is applied to the corneal surface 105 so as to treat a dry-eye condition of the eye 104. In embodiments, in response to a determination at STEP 207 that the corneal surface 105 is to be prepared for an ocular surgery, the method 200 may proceed to STEP 208.

At STEP 208 an anesthetic agent 190 may be applied to the eye 104, and at STEP 210 the eyelid 111 may be retracted and/or held by an eyelid speculum 160 so as to hold the eye 104 open.

At STEP 212, moisture may be wicked from the corneal surface 105, including by way of a sponge 150 and/or an anesthetic-loaded sponge 152, so as to develop a native developed-tackiness interface 136 at the corneal epithelium 107 after a period of time (e.g., anywhere between 20-120 seconds, such as approximately between 15 and 25 seconds, approximately between 25 and 35 seconds, approximately between 35 and 45 seconds, approximately between 45 and 55 seconds, approximately between 55 and 65 seconds, approximately between 65 and 75 seconds, approximately between 75 and 85 seconds, approximately between 85 and 95 seconds, approximately between 95 and 105 seconds, approximately between 105 and 115 seconds, approximately between 115 and 125 seconds, and/or in some cases greater than 125 seconds). In embodiments, the native developed-tackiness interface 136 may be developed at STEP 212 by a removal of surface moisture from the corneal surface 105, and the corneal surface 105 may be prepared in this manner without an application of a saline and/or a rehydrating solution to the corneal surface 105.

At STEP 214 the amniotic tissue body 102 may be laid onto the corneal surface 105 such that the amniotic tissue body 102 conforms to and fills the one or more surface irregularities 113 of the corneal surface 105 so as to present a smooth ocular surface, the amniotic tissue body 102 being retained against the corneal surface 105 by the native developed-tackiness interface 136. In embodiments, the amniotic tissue body 102 may be laid onto the corneal surface 105 at STEP 214 without debriding and/or scraping the corneal surface 105 and/or the corneal epithelium 107, as a non-site-specific body that is not configured for a particular site of the corneal surface 105, and/or without orienting a stromal side of the amniotic tissue body 102 toward the cornea, as enabled by a bilaterally symmetric laminate 110 and/or other non-handed constructions of the amniotic tissue body 102 described herein.

At STEP 216 the amniotic tissue body 102 may be smoothed and evened against the corneal surface 105 with a sponge 150, and may be secured against the corneal surface 105 by a bandage contact lens 106 placed over the amniotic tissue body 102 and/or adhered to the corneal surface 105 by way of the native developed-tackiness interface 136 and/or an applied adhesive interface 134.

At STEP 218 the amniotic tissue body 102 may be allowed to dwell upon the corneal surface 105 over a dwell period, and a recovery of the corneal surface 105 may be monitored and a follow-up assessment of the corneal surface 105 may be performed, including by way of a sodium fluorescein staining 196 and/or a photographic and/or microscopic documentation 198 of the corneal surface 105. At STEP 220, in response to a determination that the follow-up assessment does not satisfy a readiness criterion, a device-driven action may be performed upon the corneal surface 105, the device-driven action comprising repeating the method 200 and/or applying an amniotic suspension formulation 182 to the corneal surface 105 in accordance with an application procedure 430. It is noted that the device-driven action performed at STEP 220 is performed in response to the follow-up assessment, and that the device-driven action is triggered by the follow-up assessment of the corneal surface 105.

At STEP 222, in response to a determination that the corneal surface 105 satisfies the readiness criterion, one or more biometric measurements may be obtained from the corneal surface 105 and/or the eye 104, an intraocular lens may be selected based upon the one or more biometric measurements, and a surgical plan may be finalized based upon the one or more biometric measurements and/or the selected intraocular lens.

In embodiments, the steps of the method 200 may be keyed to the operative sequences described herein, such that the developing of the native developed-tackiness interface 136 at STEP 212, the laying and conforming of the amniotic tissue body 102 at STEP 214, and the smoothing and securing of the amniotic tissue body 102 at STEP 216 may be performed as described with reference to FIGS. 6A-6E, the resurfacing of the corneal surface 105 to present a smooth ocular surface may be performed as described with reference to FIGS. 5A and 5B, and an alternative delivery of an amniotic tissue to the corneal surface 105 at STEP 214 and/or STEP 220 may be performed as described with reference to FIGS. 7A and 7B.

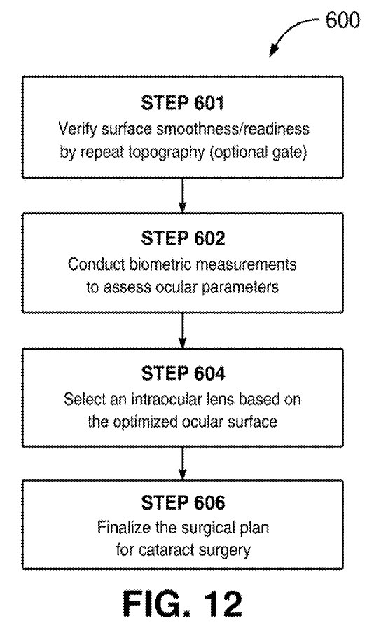

In embodiments, one or more steps of the method 200 may be performed in a different order, repeated, combined with one another, and/or omitted, and the method 200 may be performed with and/or without the branch determination at STEP 207, the verification described with reference to FIG. 12, and/or one or more of the diagnostic adjuncts described herein. It is noted that the steps and the order of the steps of the method 200 are exemplary and should not be construed as limiting in any way.

FIG. 9 shows a high-level flow diagram of a diagnosis and evaluation phase 300 for evaluating a condition of the corneal surface 105 in accordance with embodiments of the present disclosure. In embodiments, the diagnosis and evaluation phase 300 may be configured to inform a readiness of the corneal surface 105 for resurfacing by the ocular surface resurfacing system 100 and/or for a biometric measurement obtained prior to an ocular surgery.

At STEP 302 a patient questionnaire 302 may be administered, and at STEP 304 a clinical examination 304 of the corneal surface 105 may be performed so as to evaluate one or more corneal surface irregularities 113 and/or a dryness of the corneal surface 105. At STEP 306, a readiness and/or a severity associated with the corneal surface 105 may be assessed based upon the patient questionnaire 302 and/or the clinical examination 304, so as to inform whether the corneal surface 105 may be resurfaced by the ocular surface resurfacing system 100.

In embodiments, the diagnosis and evaluation phase 300 may be performed with and/or without the patient questionnaire 302, and may employ a corneal imaging and/or topography device 192 and/or a biometric measurement device 194 in connection with the clinical examination 304. It is noted that the steps and the order of the steps of the diagnosis and evaluation phase 300 are exemplary and should not be construed as limiting in any way.

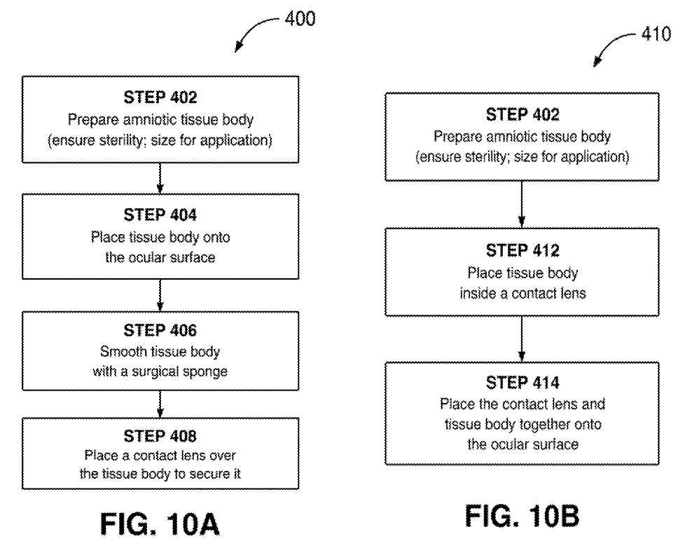

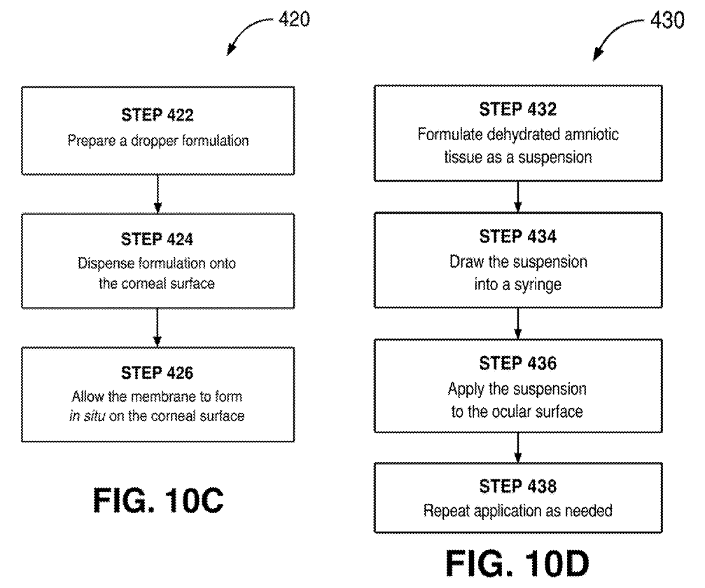

FIGS. 10A-10D show high-level flow diagrams of application procedures 400, 410, 420, and 430, respectively, for applying an amniotic tissue to the corneal surface 105 in accordance with embodiments of the present disclosure. In embodiments, the application procedures 400, 410, 420, and 430 may be configured to correspond to the modes of delivery described herein, and may be selected depending upon a clinical need.

In embodiments, as shown in FIG. 10A, the application procedure 400 may comprise preparing the amniotic tissue body 102 at STEP 402, including by providing the amniotic tissue body 102 in a sterile condition and/or sizing the amniotic tissue body 102 to the corneal surface 105, placing the amniotic tissue body 102 onto the corneal surface 105 at STEP 404, smoothing the amniotic tissue body 102 against the corneal surface 105 with a sponge 150 at STEP 406, and securing the amniotic tissue body 102 against the corneal surface 105 with a bandage contact lens 106 placed over the amniotic tissue body 102 at STEP 408.

In embodiments, as shown in FIG. 10B, the application procedure 410 may comprise preparing the amniotic tissue body 102 at STEP 411, placing the amniotic tissue body 102 within a bandage contact lens 106 so as to form an enclosing assembly 132 at STEP 412, and placing the enclosing assembly 132 onto the eye 104 such that the amniotic tissue body 102 and the bandage contact lens 106 are placed together onto the corneal surface 105 at STEP 414.

In embodiments, as shown in FIG. 10C, the application procedure 420 may comprise preparing a dropper formulation at STEP 422, dispensing the dropper formulation onto the corneal surface 105 with a dropper 170 at STEP 424, and forming an in-situ membrane 116 upon the corneal surface 105 from the dispensed dropper formulation at STEP 426.

In embodiments, as shown in FIG. 10D, the application procedure 430 may comprise formulating an amniotic suspension formulation 182 at STEP 432, drawing the amniotic suspension formulation 182 into a syringe 180 at STEP 434, applying the amniotic suspension formulation 182 to the corneal surface 105 with the syringe 180 at STEP 436, and repeating the application of the amniotic suspension formulation 182 to the corneal surface 105 as needed at STEP 438.

In embodiments, the application procedures 400, 410, 420, and 430 may be combined with one another, such that an amniotic tissue may be applied to the corneal surface 105 by a combination of two or more of the application procedures 400, 410, 420, and 430. It is noted that the application procedures and the steps thereof are exemplary and should not be construed as limiting in any way.

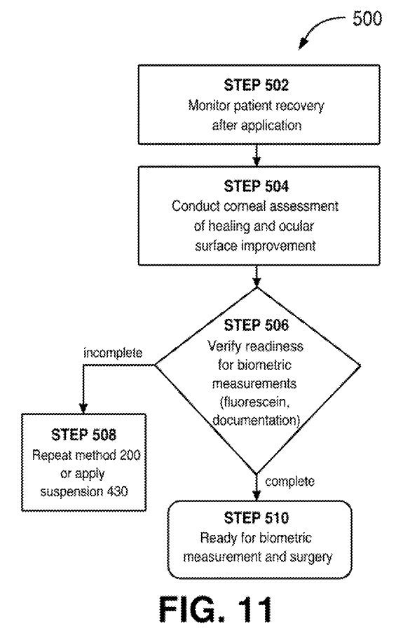

FIG. 11 shows a high-level flow diagram of a follow-up and evaluation process 500 for evaluating a condition of the corneal surface 105 following application of an amniotic tissue in accordance with embodiments of the present disclosure. In embodiments, the follow-up and evaluation process 500 may be configured to inform a readiness of the corneal surface 105 for a biometric measurement and/or an ocular surgery following a dwell period.

At STEP 502 a recovery of the corneal surface 105 may be monitored over a dwell period, and at STEP 504 a corneal assessment 504 of the corneal surface 105 may be performed, including by way of a sodium fluorescein staining 196 and/or a photographic and/or microscopic documentation 198 of the corneal surface 105. In embodiments, the corneal assessment 504 may be configured to inform a re-epithelialization and/or a centering of the amniotic tissue body 102 at the corneal surface 105.

At STEP 506 a readiness of the corneal surface 105 may be determined based upon the corneal assessment 504. At STEP 508, in response to a determination that the corneal assessment 504 does not satisfy a readiness criterion, a device-driven action may be performed upon the corneal surface 105, the device-driven action comprising repeating a method 200 of applying the amniotic tissue body 102 to the corneal surface 105 and/or applying an amniotic suspension formulation 182 to the corneal surface 105 in accordance with an application procedure 430. It is noted that the device-driven action performed at STEP 508 is performed in response to the corneal assessment 504, and that the ocular surface resurfacing system 100 is configured to perform the device-driven action upon the corneal surface 105 as triggered by the corneal assessment 504.

At STEP 510, in response to a determination that the corneal assessment 504 satisfies the readiness criterion, the corneal surface 105 may be determined to be ready for a biometric measurement and/or an ocular surgery.

In embodiments, the follow-up and evaluation process 500 may be performed over one or more dwell periods and may employ the sodium fluorescein staining 196, the photographic and/or microscopic documentation 198, a corneal imaging and/or topography device 192, and/or a biometric measurement device 194 in connection with the corneal assessment 504. It is noted that the steps and the order of the steps of the follow-up and evaluation process 500 are exemplary and should not be construed as limiting in any way.

FIG. 12 is a high-level flow diagram of a pre-surgical biometric optimization phase 600 for optimizing the corneal surface 105 for a biometric measurement prior to an ocular surgery in accordance with embodiments of the present disclosure. In embodiments, the pre-surgical biometric optimization phase 600 may be configured to obtain a biometric measurement from the corneal surface 105 following resurfacing of the corneal surface 105 by the ocular surface resurfacing system 100.

At STEP 601 a verification-by-measurement gate may be performed, the verification-by-measurement gate comprising obtaining a repeat corneal topography and/or a wavefront measurement of the corneal surface 105 so as to confirm that the corneal surface 105 satisfies a smoothing and/or a readiness criterion prior to obtaining a biometric measurement. By confirming that the corneal surface 105 satisfies the smoothing and/or the readiness criterion before obtaining the biometric measurement, the pre-surgical biometric optimization phase 600 may be configured to operate as a verification gate that closes a loop between the resurfacing of the corneal surface 105 and an accuracy of the biometric measurement.

At STEP 602 one or more biometric measurements 602 may be obtained from the corneal surface 105 and/or the eye 104; at STEP 604 an intraocular lens 604 may be selected based upon the one or more biometric measurements 602; and at STEP 606 a surgical plan 606 may be finalized based upon the one or more biometric measurements 602 and/or the selected intraocular lens 604.

In embodiments, the verification-by-measurement gate at STEP 601 may be performed and/or omitted depending upon a clinical need, and the pre-surgical biometric optimization phase 600 may employ a corneal imaging and/or topography device 192 and/or a biometric measurement device 194 in connection with the one or more biometric measurements 602. It is noted that the steps and the order of the steps of the pre-surgical biometric optimization phase 600 are exemplary and should not be construed as limiting in any way.

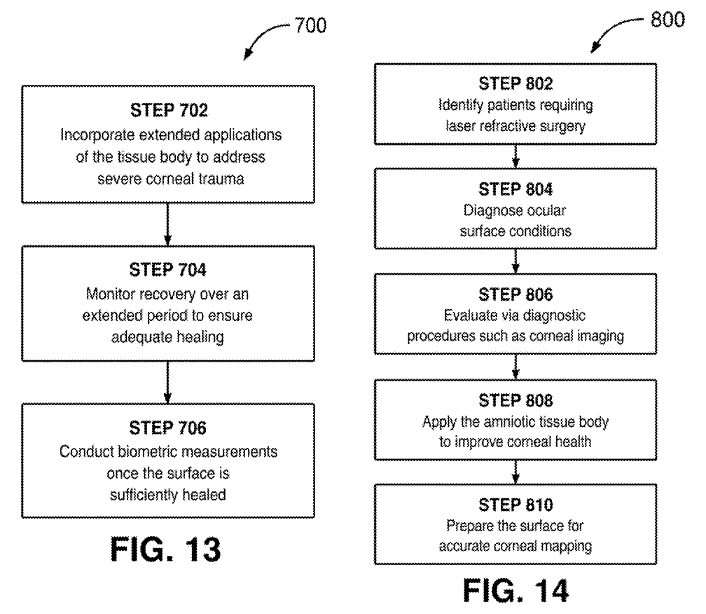

FIG. 13 is a high-level flow diagram of an application process 700 for applying an amniotic tissue to a corneal surface 105 having a corneal trauma in accordance with embodiments of the present disclosure. In embodiments, corneal trauma may include corneal abrasions, foreign object damage and/or blunt trauma (e.g., fist, ball, airbag, etc.), chemical burns, heat burns, thermal burns, ultraviolet (UV) burns, damage to the eyes with obvious external indication (e.g., lacerations, scars, scratches, etc.), damage to the eyes without obvious external indication (e.g., lacerations, scars, scratches, etc.), contact lens related trauma, surgical trauma, erosions, penetrating injuries, and/or any other type of traumatic injury to the corneal surface.

In embodiments, the application process 700 may be configured to apply the amniotic tissue body 102 to the corneal surface 105 over an extended application in connection with a corneal trauma of the corneal surface 105. For example, at STEP 702 the amniotic tissue body 102 may be applied to the corneal surface 105 over an extended application configured for a corneal trauma of the corneal surface 105. In embodiments, an extended application may include applying the amniotic tissue body 102 for an extended period of time (e.g., any time between 1-10 weeks, and any range therebetween).

At STEP 704 a recovery of the corneal surface 105 may be monitored over an extended monitoring period. At STEP 706 a biometric measurement may be obtained from the corneal surface 105 once the corneal surface 105 has healed.

In embodiments, the severe-trauma application process 700 may comprise one or more repeated and/or extended applications of the amniotic tissue body 102 to the corneal surface 105 over one or more dwell periods depending upon a severity of the severe corneal trauma. It is noted that the steps and the order of the steps of the severe-trauma application process 700 are exemplary and should not be construed as limiting in any way.

FIG. 14 is a high-level flow diagram of a laser-refractive preoperative preparation process 800 for preparing a corneal surface 105 for a laser-refractive procedure in accordance with embodiments of the present disclosure. In embodiments, the laser-refractive preoperative preparation process 800 may be configured to prepare the corneal surface 105 for an accurate corneal mapping prior to a laser-refractive procedure.

At STEP 802 a patient for a laser-refractive procedure may be identified, at STEP 804 a condition of the corneal surface 105 may be diagnosed, at STEP 806 the corneal surface 105 may be evaluated by way of a corneal imaging and/or topography device 192, at STEP 808 the amniotic tissue body 102 may be applied to the corneal surface 105 so as to improve a corneal health of the corneal surface 105, and at STEP 810 the corneal surface 105 may be prepared for an accurate corneal mapping prior to the laser-refractive procedure.

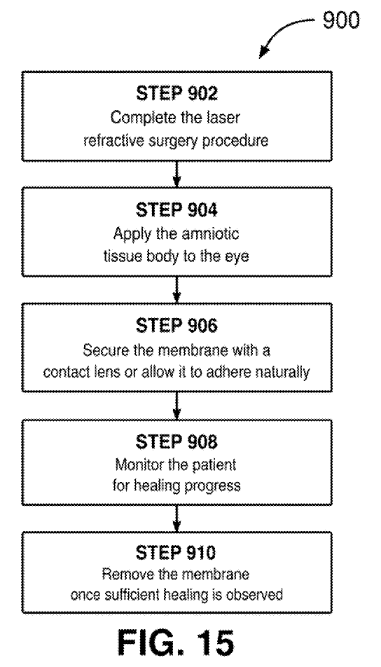

FIG. 15 is a high-level flow diagram of a laser-refractive postoperative corneal healing process 900 for promoting a healing of a corneal surface 105 following a laser-refractive procedure in accordance with embodiments of the present disclosure. In embodiments, the laser-refractive postoperative corneal healing process 900 may be configured to apply the amniotic tissue body 102 to the corneal surface 105 so as to promote a healing of the corneal surface 105 following a laser-refractive procedure.

At STEP 902 a laser-refractive procedure may be completed, at STEP 904 the amniotic tissue body 102 may be applied to the corneal surface 105, at STEP 906 the amniotic tissue body 102 may be secured against the corneal surface 105 by a bandage contact lens 106 and/or by an adhesive interface 134 and/or a native developed-tackiness interface 136, at STEP 908 a healing of the corneal surface 105 may be monitored, and at STEP 910 the amniotic tissue body 102 may be removed from the corneal surface 105 once the corneal surface 105 has healed.

In embodiments, the regenerative and/or anti-inflammatory factors of the amniotic tissue body 102 may be adapted to a laser-refractive context in a preoperative sub-context and/or a postoperative sub-context. For example, in a preoperative sub-context, the amniotic tissue body 102 may be configured to improve a corneal health of the corneal surface 105 so as to support an accurate corneal mapping prior to a laser-refractive procedure, as described with reference to FIG. 14, and, in a postoperative sub-context, the amniotic tissue body 102 may be configured to promote a healing of the corneal surface 105 following a laser-refractive procedure so as to reduce a haze and/or a regression associated with the corneal surface 105, as described with reference to FIG. 15. By adapting the same regenerative and/or anti-inflammatory factors of the amniotic tissue body 102 to the preoperative sub-context and the postoperative sub-context, the ocular surface resurfacing system 100 may be configured for use across a laser-refractive context.

In embodiments, the laser-refractive preoperative preparation process 800 and the laser-refractive postoperative corneal healing process 900 may be performed independently of one another and/or in connection with one another for a patient undergoing a laser-refractive procedure. It is noted that the steps and the order of the steps of the laser-refractive preoperative preparation process 800 and the laser-refractive postoperative corneal healing process 900 are exemplary and should not be construed as limiting in any way.

In embodiments, the ocular surface resurfacing system 100 and the methods of using the same described herein may incorporate one or more additional considerations and/or alternative embodiments, as described herein. It is noted that the considerations and embodiments described herein may be employed in connection with any of the embodiments described herein.

In embodiments, an anesthetic agent 190 may be applied to the eye 104 in connection with any of the procedures described herein for patient comfort, and an eyelid speculum 160 may be employed to retract the eyelid 111 and to hold the eye 104 open, including where a blinking of the eye 104 may otherwise impede placement of the amniotic tissue body 102 upon the corneal surface 105.

In embodiments, the amniotic tissue body 102 may be subjected to a thermal conditioning configured to improve a conformance of the amniotic tissue body 102 to the corneal surface 105, and/or to a controlled rehydration with a defined solution configured to tune a conformability of the amniotic tissue body 102 to a target conformability prior to laying the amniotic tissue body 102 onto the corneal surface 105.

In embodiments, the amniotic tissue body 102 may be sized and/or cut to a contour derived from a corneal topography and/or an irregularity map of the corneal surface 105 obtained by a corneal imaging and/or topography device 192, and/or may be sized to one or more coverage targets of the corneal surface 105. In embodiments, the amniotic tissue body 102 may be provided as a family of sizes supplied as a kit, and may present a non-handed plan-view outline lacking a handedness indicator.

In embodiments, a centration and/or a coverage of the amniotic tissue body 102 upon the corneal surface 105 may be confirmed by way of a dye and/or a fiducial, and/or by way of a photographic and/or microscopic documentation 198 of the corneal surface 105.

In embodiments, a punctal plug may be placed in a punctum of the eye 104 so as to provide an adjunct retention of a tear film and/or of one or more therapeutic factors at the corneal surface 105 over a dwell period.

In embodiments, the corneal surface 105 may be charged so as to attract an amniotic suspension formulation 182 to the corneal surface 105, and/or the amniotic suspension formulation 182 may be taken up at the corneal surface 105 by way of an iontophoresis-assisted uptake, so as to increase a bioavailability of the amniotic suspension formulation 182 at the corneal surface 105.

In embodiments, the amniotic tissue body 102 and a contact lens may be combined into a single article so as to form a hybrid lens-and-membrane assembly, in which the amniotic tissue body 102 is incorporated with the contact lens as a unitary article configured to be placed upon the corneal surface 105. In embodiments, the hybrid lens-and-membrane assembly may be configured to retain the amniotic tissue body 102 against the corneal surface 105 and/or to deliver one or more therapeutic factors to the corneal surface 105, in a manner of the enclosing assembly 132 and/or the therapeutic-eluting lens 140 described herein, and may be provided as a manufacturer-prepared article so as to facilitate application of the amniotic tissue body 102 to the corneal surface 105.

In embodiments, a procedure described herein may be associated with one or more billing codes for documentation and/or reimbursement purposes. It is noted that, in one non-limiting example, a procedure described herein may be associated with one or more CPT codes, such as CPT code 65778, CPT code 92071, and/or CPT code 92285, and that any such billing codes are provided as exemplary context and should not be construed as limiting in any way.

It is noted that in some embodiments, application of an anesthetic agent may be performed when the process or method includes placing a tissue body onto the ocular surface directly (e.g., FIGS. 10A and 10B. In these cases, and in some embodiments, the application of an anesthetic agent may be mandatory to prevent discomfort to the patient. On the other hand, when a process includes application of a suspension (e.g., FIGS. 10C and 10D), the application of an anesthetic agent may be optional.

In embodiments, the components, instruments, agents, procedures, and steps described herein may be employed in any suitable combination, and one or more of the components, instruments, agents, procedures, and/or steps described herein may be omitted, such that the embodiments described herein may be used in combination with one another and/or separately from one another. It is noted that the foregoing components, instruments, agents, procedures, steps, combinations, and omissions are exemplary and should not be construed as limiting in any way.

What is claimed is:

1. An ocular surface resurfacing system, comprising:
an amniotic tissue body configured to be laid onto a corneal surface of an eye and to conform to and fill one or more surface irregularities of the corneal surface so as to present a smooth ocular surface, the amniotic tissue body comprising a non-handed construction configured to present equivalent amniotic tissue to the corneal surface independent of which face of the amniotic tissue body is placed against the corneal surface; and
a securing element configured to retain the amniotic tissue body against the corneal surface, wherein the ocular surface resurfacing system is configured to resurface the corneal surface without debriding or scraping the corneal surface or a corneal epithelium thereof, such that the corneal epithelium is preserved beneath the amniotic tissue body.

2. The ocular surface resurfacing system of claim 1, wherein the non-handed construction includes a bilaterally symmetric laminate comprising two layers of amniotic tissue bonded at a central interface, wherein an outwardly-presenting surface of each of the two layers defines a respective equivalent first face and second face of the bilaterally symmetric laminate.

3. The ocular surface resurfacing system of claim 1, wherein the non-handed construction includes one or more of:
a homogenized or reconstituted non-handed sheet lacking a layered handedness;
an in-situ membrane formed upon the corneal surface from a dispensed formulation; and
a suspension form, each configured to present equivalent amniotic tissue to the corneal surface independent of a placement orientation.

4. The ocular surface resurfacing system of claim 1, wherein the amniotic tissue body includes a cryopreserved amniotic tissue, a dehydrated amniotic tissue, or a hydrated amniotic tissue, and is cut to fit the corneal surface from a non-site-specific starting stock.

5. The ocular surface resurfacing system of claim 1, wherein the securing element includes one or more of:
a bandage contact lens configured to be placed over the amniotic tissue body;
an enclosing assembly in which the amniotic tissue body is held within a bandage contact lens;
an applied adhesive interface disposed on the amniotic tissue body or the corneal surface; and
a fibrin interface disposed between the amniotic tissue body and the corneal surface.

6. The ocular surface resurfacing system of claim 1, wherein the securing element includes a native developed-tackiness interface formed by removing moisture from the corneal surface such that the corneal epithelium develops a tacky consistency by which the amniotic tissue body is retained against the corneal surface without an application of a saline or a rehydrating solution to the corneal surface.

7. The ocular surface resurfacing system of claim 1, wherein the securing element includes a therapeutic-eluting lens configured to retain the amniotic tissue body against the corneal surface and to deliver one or more therapeutic factors to the corneal surface, the therapeutic-eluting lens comprising at least one of amniotic stem cells embedded within a matrix of the therapeutic-eluting lens, an amniotic material absorbed into the therapeutic-eluting lens from a soaking solution, or an adjunct load comprising a hyaluronic acid, an autologous serum or a platelet-rich plasma, or a steroid.

8. The ocular surface resurfacing system of claim 1, wherein the amniotic tissue body includes a multilayer stack of two or more bodies of amniotic tissue, the two or more bodies comprising amniotic tissues of two or more different tissue types.

9. The ocular surface resurfacing system of claim 1, wherein the amniotic tissue body is sized to a contour derived from a corneal topography or an irregularity map of the corneal surface, or is provided as a family of sizes supplied as a kit.

10. The ocular surface resurfacing system of claim 1, further comprising one or more of:
a sponge configured to wick moisture from the corneal surface or to smooth the amniotic tissue body against the corneal surface;
an eyelid speculum configured to hold the eye open;
a dropper configured to dispense a formulation that forms an in-situ membrane upon the corneal surface;
a syringe configured to apply an amniotic suspension formulation to the corneal surface; and
an anesthetic agent configured to be applied to the eye.

11. A method of preparing a corneal surface of an eye for an ocular surgery, the method comprising:
developing a native tackiness of the corneal surface by removing moisture from the corneal surface, without applying a saline or a rehydrating solution to the corneal surface, such that a corneal epithelium of the corneal surface develops a tacky consistency;
laying an amniotic tissue body onto the corneal surface such that the amniotic tissue body conforms to and fills one or more surface irregularities of the corneal surface so as to present a smooth ocular surface, the amniotic tissue body comprising a non-handed construction that presents equivalent amniotic tissue to the corneal surface independent of which face of the amniotic tissue body is placed against the corneal surface, and the amniotic tissue body being laid onto the corneal surface without orienting a stromal side of the amniotic tissue body toward the corneal surface and without debriding or scraping the corneal surface or the corneal epithelium, such that the corneal epithelium is preserved beneath the amniotic tissue body;
retaining the amniotic tissue body against the corneal surface, at least in part by the tacky consistency of the corneal epithelium or by a securing element placed over the amniotic tissue body;
allowing the amniotic tissue body to dwell upon the corneal surface over a dwell period during which a regenerative or anti-inflammatory factor of the amniotic tissue body is delivered to the corneal surface; and
obtaining a biometric measurement or a corneal mapping from the smooth ocular surface presented by the amniotic tissue body for the ocular surgery.

12. The method of claim 11, further comprising:
applying an anesthetic agent to the eye and retracting an eyelid of the eye with an eyelid speculum so as to hold the eye open during the laying of the amniotic tissue body onto the corneal surface.

13. The method of claim 11, wherein developing the native tackiness of the corneal surface comprises wicking moisture from the corneal surface with a sponge over a period of time sufficient for the corneal epithelium to develop the tacky consistency, the period of time being in a range of approximately 40 to 80 seconds.

14. The method of claim 11, further comprising:
smoothing the amniotic tissue body against the corneal surface with a sponge prior to or during the retaining of the amniotic tissue body against the corneal surface.

15. The method of claim 11, wherein retaining the amniotic tissue body against the corneal surface comprises one or more of:
placing a bandage contact lens over the amniotic tissue body;
placing the amniotic tissue body within a bandage contact lens and placing the amniotic tissue body and the bandage contact lens together onto the corneal surface;
applying an adhesive interface between the amniotic tissue body and the corneal surface; and
allowing the amniotic tissue body to be retained against the corneal surface by the tacky consistency of the corneal epithelium.

16. The method of claim 11, wherein the amniotic tissue body is provided to the corneal surface by at least one of forming an in-situ membrane upon the corneal surface from a formulation dispensed by a dropper, or applying an amniotic suspension formulation to the corneal surface with a syringe, each of the in-situ membrane and the amniotic suspension formulation comprising the non-handed construction.

17. The method of claim 11, further comprising:
performing, after the dwell period, a corneal assessment of the corneal surface, including by a sodium fluorescein staining of the corneal surface; and
performing, in response to a determination that the corneal assessment does not satisfy a readiness criterion, a device-driven action comprising one or more of:
repeating the laying of the amniotic tissue body onto the corneal surface; and
applying an amniotic suspension formulation to the corneal surface.

18. The method of claim 11, further comprising:
performing, prior to obtaining the biometric measurement or the corneal mapping, a verification-by-measurement gate comprising obtaining a repeat corneal topography or a wavefront measurement of the corneal surface to confirm that the smooth ocular surface satisfies a smoothing criterion.

19. The method of claim 11, wherein the ocular surgery comprises a cataract surgery and the biometric measurement is obtained from the smooth ocular surface, the method further comprising:
selecting an intraocular lens based on the biometric measurement and finalizing a surgical plan for the cataract surgery based on the biometric measurement or the selected intraocular lens.

20. The method of claim 11, wherein the ocular surgery comprises a laser-refractive procedure and the corneal mapping is obtained from the smooth ocular surface for the laser-refractive procedure, the method further comprising:
applying the amniotic tissue body to the corneal surface following the laser-refractive procedure so as to promote a healing of the corneal surface.
